(19) United States
(12) Patent Application Publication
(10) Pub. No.: US 2018/0071221 A1
(43) Pub. Date: Mar. 15, 2018

(54) IMMEDIATE RELEASE SOLUBLE IBUPROFEN COMPOSITIONS

(71) Applicant: BIONPHARMA HEALTHCARE LLC, PRINCETON, NJ (US)

(72) Inventors: Tanesha ROBERTS, Greensboro, NC (US); Qi FANG, Oak Ridge, NC (US); Tatyana DYAKONOV, Greensboro, NC (US); Aqeel A. FATMI, High Point, NC (US); Saujanya GOSANGARI, Jamestown, NC (US); Peijin ZHANG, Oak Ridge, NC (US)

(21) Appl. No.: 15/554,542

(22) PCT Filed: Mar. 1, 2016
(86) PCT No.: PCT/US16/20158

§ 371 Date: Aug. 30, 2017

Related U.S. Application Data
(60) Provisional application No. 62/127,002, filed on Mar. 2, 2015.

(51) Int. Cl. A61K 9/48; A61K 31/192; A61K 45/06

(52) U.S. Cl. CPC A61K 9/4858; A61K 9/4866; A61K 2300/00; A61K 45/06; A61K 31/192

(57) ABSTRACT

Described herein are pharmaceutical compositions comprising ibuprofen, ibuprofen salts, or combinations thereof, methods for making the same, and methods for treating subjects in need thereof. In particular, there is described, inter alia, immediate release oral pharmaceutical compositions comprising ionic forms of ibuprofen are described.

### PRIORITY

This application claims the benefit of priority of U.S. Patent Application No. 62/127,002, filed Mar. 2, 2015, the entire contents of all of which are incorporated by reference herein

### TECHNICAL FIELD

Described herein are pharmaceutical compositions comprising ibuprofen, ibuprofen salts, or combinations thereof, methods for making the same, and methods for treating subjects in need thereof. In particular, there is described, inter alia, immediate release oral pharmaceutical compositions comprising ionic forms of ibuprofen are described.

### BACKGROUND

The bioavailability of a drug depends on several factors, including drug solubility in an aqueous environment and drug permeability through lipophilic membranes. Orally administered drugs absorb and show good bioavailability only when they are soluble in the gastric medium. Only solubilized drug molecules can be absorbed by the cellular membranes to subsequently reach the site of drug action. Any drug, to be absorbed, must be present in the form of an aqueous solution before it is absorbed from the intestinal tract into the bloodstream.

Pain and acute pain, in particular, requires a rapid onset of relief, which is an important attribute of over the counter and prescription analgesics. Ibuprofen sodium dihydrate is a salt of ibuprofen that is highly soluble in the stomach and rapidly absorbed, which in turn provides faster and greater pain relief than ibuprofen free acid.

Ibuprofen (IBU) is a widely used, safe, and effective non-steroidal anti-inflammatory drug (NSAID) that has analgesic, anti-inflammatory, and antipyretic activity. The effects of IBU can be directly correlated with IBU blood plasma levels. Even though nearly all (˜100%) of IBU is absorbed, the rate at which it is absorbed is dependent upon the formulation. IBU in its free acid form is a carboxylic acid, which has a low solubility in acidic pH gastric environments. Thus, any perceptible pain relief from such formulations can take up to 45 minutes or longer following ingestion.

Second generation IBU formulations have been contemplated to decrease the time at which pain relief can be perceived. For example, IBU has been formulated as various salt conjugates, such as IBU lysinate, arginine, or sodium and as a liquid in gelatin capsules. Each of these formulations provides increased maximum plasma concentration (Cₘₐₓ) and a decreased time to maximal therapeutic efficacy (Tₘₐₓ); ibuprofen sodium dihydrate provides the most rapid Tₘₐₓ. See, Schleier et al. Int. J. Clin. Pharmacol. Theor. 45(2): 89-97 (2007) and Legg et al, Drugs in R & D, 14: 283-290 (2014), each of which is incorporated by reference herein for such teachings. Yet, ibuprofen sodium has only been successfully formulated as a film coated caplet or tablet.

There are numerous well-known problems associated with film coatings including large variations from tablet to tablet from too little coating, tablet twinning during processing, cracking, peeling, orange peel (roughness), tablet core erosion and tablet chipping, to name a few. Furthermore, patient compliance is often decreased, particularly in the elderly, due to tablet “stickiness” and problems swallowing.

Soft capsules have gained increased popularity and acceptance due to their elegant and clear gelatin shell. Furthermore, soft capsules are uniform, stable, dissolve quickly, allow for liquid formulations, and are easier to swallow.

Thus, there is an unmet need for a soft capsule immediate release ibuprofen formulation that is as fast as or faster than currently available tablet and caplet forms. The formulation, would overcome limitations of poor aqueous solubility, be highly stable, have increased bioavailability, provide rapid perceptible relief onset, and reduce plasma level variability. Accordingly, it is desirable to develop immediate release formulations of ibuprofen sodium in soft gelatin capsules.

### SUMMARY

One embodiment described herein is an immediate release pharmaceutical composition comprising a soft capsule shell encapsulating a matrix fill comprising an ionic form of ibuprofen.

Another embodiment described herein is an oral immediate release pharmaceutical composition comprising a soft capsule encapsulating a matrix comprising: (a) at least one hydrophilic vehicle; (b) at least one co-solvent; (c) at least one pH modifier; and (d) one or more active pharmaceutical ingredients; wherein the ratio of the active pharmaceutical ingredient to the pH modifier is about 3.5:1 to about 5.5:1. In another embodiment, the composition releases substantially all of the one or more active pharmaceutical ingredients after about 20 minutes in vitro.

In another embodiment described herein, the matrix comprises: (a) about 30-70% of at least one hydrophilic vehicle; (b) about 3-5% of at least one co-solvent; (c) about 3-10% of at least one pH modifier; and (d) about 20-40% of one or more active pharmaceutical ingredients. In one aspect described herein, the hydrophilic vehicle comprises one or more polyethylene glycols. In another aspect described herein, the co-solvent comprises propylene glycol, glycerol, or a combination thereof. In another aspect described herein, the pH modifier comprises acetic acid, lactic acid, malic acid, citric acid, tartaric acid, or a combination thereof. In another aspect described herein, the pH modifier comprises lactic acid. In another aspect described herein, the composition comprises a pH modifier comprising lactic acid at a weight percentage up to about 8%. In another aspect described herein, the active pharmaceutical ingredient comprises one or more of aspirin, ibuprofen, aceclofenac, acemetacin, aloxiprin, azapropazone, benorilate, bromfenac, carprofen, celecoxib, choline magnesium salicylate, diclofenac, diflunisal, etodolac, etoricoxib, faislamine, fenbufen, fenoprofen, flurbiprofen, indometacin, ketoprofen, ketorolac, lornoxicam, loxoprofen, meloxicam, meclofenamic acid, mefenamic acid, meloxicam, metamizole, methyl salicylate, magnesium salicylate, nabumetone, naproxen, nimesulide, oxyphenbutazone, parecoxib, phenylbutazone, piroxicam, salicyl salicylate, sulindac, sulfinpyrazone, suprofen, tenoxicam, tiaprofenic acid, tolmetin, valdecoxib, salts thereof, or combinations thereof. In another aspect described herein, the active pharmaceutical ingredient comprises a salt form of ibuprofen. In another aspect described herein, the active pharmaceutical ingredient comprises about 260 mg of ibuprofen sodium. In another aspect described herein, the ratio of the active pharmaceutical ingredient to the pH modifier is about 5:1. In another aspect described herein, the ratio of the active pharmaceutical ingredient to hydrophilic vehicle and co-solvent is about 1:2 to about 1:4. In another aspect described herein, the ratio of the active pharmaceutical ingredient to a combined weight percentage of the hydrophilic vehicle, co-solvent, and pH modifier is about 1:2 to about 1:3.

In another embodiment described herein, the active pharmaceutical ingredient comprises a salt form of ibuprofen and one or more of a cold, cough, allergy, stimulant, sedative, anti-inflammatory, antibiotic, anti-viral, anti-asthmatic, anti-migraine, hypnotic, narcotic analgesic, or narcotic antagonist active pharmaceutical ingredients. In another aspect described herein, the active pharmaceutical ingredient comprises a salt form of ibuprofen and one or more of astemizole, azelastine, azatadine, brompheniramine, carbinoxamine, cetirizine, chlorpheniramine, clemastine, cyproheptadine, desloratadine, dexbrompheniramine, dexchlorpheniramine, diphenhydramine, fexofenadine, hydroxyzine, levocetirizine, loratadine, phenindamine, pheniramine, phenyltoloxamine, promethazine, pyrilamine, terfenadine, tripelennamine, triprolidine, acetyl dihydrocodeine, benproperine, benzonatate, benzylmorphine, bibenzonium bromide, butamirate, butorphanol, carbetapentane, chlophedianol, clobutinol, clofedanol, cloperastine, codeine, dextromethorphan, diacetylmorphine, dibunate, dihydrocodeine, dimemorfan, dimethoxanate, diphenhydramine, dropropizine, droxypropine, ethylmorphine, fedrilate, glaucine, hydrocodone, hydromorphone, isoaminile, laudanum, levodropropizine, levomethadone, levopropoxyphene, meprotixol, methadone, morclofone, nepinalone, nicocodine, nicodicodine, normethadone, noscapine, oxeladin, oxolamine, pentoxyverine, pholcodine, pipazetate, piperidione, prenoxdiazine, tipepidine, zipeprol, acetylcysteine, althea root, ambroxol, antimony pentasulfide, bromhexine, carbocisteine, cineole, combinations, combinations, creosote, dembrexine hydrochloride, domiodol, dornase alfa, eprazinone, erdosteine, guaiacolsulfonate, guaifenesin, hederae helicis folium, ipecacuanha, letosteine, levo verbenone, mannitol, mesna, neltenexine, potassium iodide, senega, sobrerol, stepronin, tiopronin, tyloxapol, or a mixture or combination thereof.

In another embodiment described herein, the matrix comprises: (a) about 39% polyethylene glycol 600; (b) about 23% polyethylene glycol 400; (c) about 3% propylene glycol; (d) about 6% lactic acid; and (e) about 30% ibuprofen sodium salt. In one aspect described herein, the composition comprises about 260 mg ibuprofen sodium that provides an equivalent therapeutic dose as that of about 200 mg of ibuprofen free acid.

In another embodiment described herein, the composition provides one or more of the following pharmacokinetic parameters: (a) a mean plasma ibuprofen Tₘₐₓ of about 0.7 hours to about 1.8 hours; (b) a mean plasma ibuprofen Cₘₐₓ of about 15 mg/L to about 29 mg/L; (c) a mean plasma ibuprofen AUC_{0→12 h} of about 62 h·mg/L to about 77 h·mg/L; (d) a mean plasma ibuprofen AUC_{0→∞} of about 66·mg/L to about 79·mg/L; (e) a mean ibuprofen half-life (t½) of about 2.3 h to about 2.4 h; or (f) a mean ibuprofen terminal elimination rate constant (λ_{z}) of about 0.29 h⁻¹ to about 0.31 h⁻¹.

In another embodiment described herein, the composition provides one or more of the following pharmacokinetic parameters: (a) a mean plasma ibuprofen Tₘₐₓ of about 0.7 hours under fasting conditions; (b) a mean plasma ibuprofen Cₘₐₓ of about 29 mg/L under fasting conditions; (c) a mean plasma ibuprofen AUC_{0→12 h} of about 77 h·mg/L under fasting conditions; (d) a mean plasma ibuprofen AUC_{0→∞} of about 79·mg/L under fasting conditions; (e) a mean ibuprofen half-life (t½) of about 2.4 h under fasting conditions; or (f) a mean ibuprofen terminal elimination rate constant (λ_{z}) of about 0.29 h⁻¹ under fasting conditions; or (g) a mean plasma ibuprofen Tₘₐₓ of about 1.8 hours under fed conditions; (h) a mean plasma ibuprofen Cₘₐₓ of about 15 mg/L under fed conditions; (i) a mean plasma ibuprofen AUC_{0→12 h} of about 62 h·mg/L under fed conditions; (j) a mean plasma ibuprofen AUC_{0→∞} of about 66·mg/L under fed conditions; (k) a mean ibuprofen half-life (t½) of about 2.3 h under fed conditions; or (l) a mean ibuprofen terminal elimination rate constant (λ_{z}) of about 0.31 h⁻¹ under fed conditions.

In another embodiment described herein, the composition is useful for treating, retarding the progression of, delaying the onset of, prophylaxis of, amelioration of, or reducing the symptoms of pain, inflammation, or fever. In another aspect described herein, the composition is useful for treating, retarding the progression of, delaying the onset of, prophylaxis of, amelioration of, or reducing the symptoms of pain, inflammation, or fever, including but not limited to, bacterial or viral infections, osteoarthritis rheumatoid arthritis, tendonitis, bursitis, chronic neuropathies, shingles, chronic sports injuries, chronic malignancies and/or cancer, radiculopathy, sciatica, kidney stones, menstrual pain or inflammation, dysmenorrhea, endometriosis, headache, acute migraines, ankylosing spondylitis, spondylarthritis, or gout.

In another embodiment described herein, the soft capsule comprises: (a) about 25-50% of a film-forming polymer; (b) about 15-25% of a plasticizer; (c) about 20-40% of a solvent; (d) optionally, about 0.05-0.1% of an coloring agent; and (e) optionally, about 0.5-1.5% of an opacifier.

In another embodiment described herein, the soft capsule comprises: (a) about 40% of at least one film-forming polymer; (b) about 20% of at least one plasticizer; (c) about 36% of a solvent; and (d) optionally, about 0.1% of a coloring agent.

In another embodiment described herein, the soft capsule comprises: (a) about 43% gelatin; (b) about 20% glycerol; and (c) about 36% water.

Another embodiment described herein is an immediate release pharmaceutical composition comprising a soft capsule encapsulating a matrix fill, the soft capsule comprising: (a) about 40% of at least one film-forming polymer; (b) about 20% of at least one plasticizer; (c) about 36% of a solvent; and (d) optionally, about 0.1% of a coloring agent; and the matrix fill comprising: (e) about 39% polyethylene glycol 600; (f) about 23% polyethylene glycol 400; (g) about 3% propylene glycol; (h) about 6% lactic acid; and (i) about 30% ibuprofen, sodium salt. In one embodiment described herein, the composition releases essentially all of the ibuprofen after about 20 minutes in vitro. In one aspect described herein, the ibuprofen sodium is about 260 mg.

In one embodiment described herein, the composition provides one or more of the following pharmacokinetic parameters: (a) a mean plasma ibuprofen Tₘₐₓ of about 0.7 hours to about 1.8 hours; (b) a mean plasma ibuprofen Cₘₐₓ of about 15 mg/L to about 29 mg/L; (c) a mean plasma ibuprofen AUC_{0→12 h} of about 62 h·mg/L to about 77 h·mg/L; (d) a mean plasma ibuprofen AUC_{0→∞} of about 66·mg/L to about 79·mg/L; (e) a mean ibuprofen half-life (t½) of about 2.3 h to about 2.4 h; or (f) a mean ibuprofen terminal elimination rate constant (λ_{z}) of about 0.29 h⁻¹ to about 0.31 h⁻¹.

In another embodiment described herein, the composition provides one or more of the following pharmacokinetic parameters: (a) a mean plasma ibuprofen Tₘₐₓ of about 0.7 hours under fasting conditions; (b) a mean plasma ibuprofen Cₘₐₓ of about 29 mg/L under fasting conditions; (c) a mean plasma ibuprofen AUC_{0→12 h} of about 77 h·mg/L under fasting conditions; (d) a mean plasma ibuprofen AUC_{0→∞} of about 79·mg/L under fasting conditions; (e) a mean ibuprofen half-life (t½) of about 2.4 h under fasting conditions; or (f) a mean ibuprofen terminal elimination rate constant (λ_{z}) of about 0.29 h⁻¹ under fasting conditions; or (g) a mean plasma ibuprofen Tₘₐₓ of about 1.8 hours under fed conditions; (h) a mean plasma ibuprofen Cₘₐₓ of about 15 mg/L under fed conditions; (i) a mean plasma ibuprofen AUC_{0→12 h} of about 62 h·mg/L under fed conditions; (j) a mean plasma ibuprofen AUC_{0→∞} of about 66·mg/L under fed conditions; (k) a mean ibuprofen half-life (t½) of about 2.3 h under fed conditions; or (l) a mean ibuprofen terminal elimination rate constant (λ_{z}) of about 0.31 h⁻¹ under fed conditions.

In another embodiment described herein, the composition is useful for treating, retarding the progression of, delaying the onset of, prophylaxis of, amelioration of, or reducing the symptoms of pain, inflammation, fever, or symptoms stemming from cough or cold. In one aspect described herein, the active pharmaceutical ingredient further comprises one or more of astemizole, azelastine, azatadine, brompheniramine, carbinoxamine, cetirizine, chlorpheniramine, clemastine, cyproheptadine, desloratadine, dexbrompheniramine, dexchlorpheniramine, diphenhydramine, fexofenadine, hydroxyzine, levocetirizine, loratadine, phenindamine, pheniramine, phenyltoloxamine, promethazine, pyrilamine, terfenadine, tripelennamine, triprolidine, acetyl dihydrocodeine, benproperine, benzonatate, benzylmorphine, bibenzonium bromide, butamirate, butorphanol, carbetapentane, chlophedianol, clobutinol, clofedanol, cloperastine, codeine, dextromethorphan, diacetylmorphine, dibunate, dihydrocodeine, dimemorfan, dimethoxanate, diphenhydramine, dropropizine, droxypropine, ethylmorphine, fedrilate, glaucine, hydrocodone, hydromorphone, isoaminile, laudanum, levodropropizine, levomethadone, levopropoxyphene, meprotixol, methadone, morclofone, nepinalone, nicocodine, nicodicodine, normethadone, noscapine, oxeladin, oxolamine, pentoxyverine, pholcodine, pipazetate, piperidione, prenoxdiazine, tipepidine, zipeprol, acetylcysteine, althea root, ambroxol, antimony pentasulfide, bromhexine, carbocisteine, cineole, combinations, combinations, creosote, dembrexine hydrochloride, domiodol, dornase alfa, eprazinone, erdosteine, guaiacolsulfonate, guaifenesin, hederae helicis folium, ipecacuanha, letosteine, levo verbenone, mannitol, mesna, neltenexine, potassium iodide, senega, sobrerol, stepronin, tiopronin, tyloxapol, or a mixture or combination thereof. In one aspect described herein, the composition is useful for treating, retarding the progression of, delaying the onset of, prophylaxis of, amelioration of, or reducing the symptoms of pain, inflammation, or fever, including but not limited to, bacterial or viral infections, osteoarthritis rheumatoid arthritis, tendonitis, bursitis, chronic neuropathies, shingles, chronic sports injuries, chronic malignancies and/or cancer, radiculopathy, sciatica, kidney stones, menstrual pain or inflammation, dysmenorrhea, endometriosis, headache, acute migraines, ankylosing spondylitis, spondylarthritis, or gout.

Another embodiment described herein is a method for delivering a 200 mg dose equivalent of ibuprofen free acid comprising administering to a subject ibuprofen sodium admixed with lactic acid and other pharmaceutically acceptable excipients in a soft gel capsule, the method capable of achieving one or more of the following pharmacokinetic parameters: (a) a mean plasma ibuprofen Tmax of about 0.7 hours to about 1.8 hours; (b) a mean plasma ibuprofen Cmax of about 15 mg/L to about 29 mg/L; (c) a mean plasma ibuprofen AUC0→12 h of about 62 h·mg/L to about 77 h·mg/L; (d) a mean plasma ibuprofen AUC0→∞ of about 66·mg/L to about 79·mg/L; (e) a mean ibuprofen half-life (t½) of about 2.3 h to about 2.4 h; or (f) a mean ibuprofen terminal elimination rate constant (λ_{z}) of about 0.29 h-1 to about 0.31 h-1. In another embodiment describe herein, the dose equivalent comprises about 260 mg of ibuprofen sodium. In another embodiment described herein, lactic acid comprises about 50 to about 60 mg. In another embodiment described herein, the pharmaceutically acceptable excipients comprise one or more polyethylene glycols, propylene glycol, or a combination thereof. In another embodiment described herein, the subject experiences relief from one or more of the symptoms of pain, inflammation, or fever.

Another embodiment described herein is a method for treating, retarding the progression of, delaying the onset of, prophylaxis of, amelioration of, or reducing the symptoms of pain, inflammation, fever, or symptoms stemming from cough or cold comprising the administration of a therapeutically effective amount of ibuprofen sodium comprising any one of the pharmaceutical compositions as described herein. In one aspect described herein, the therapeutically effective amount of ibuprofen sodium is about 260 mg. In another aspect described herein, administering the composition provides one or more of the following pharmacokinetic parameters: (a) a mean plasma ibuprofen Tₘₐₓ of about 0.7 hours to about 1.8 hours; (b) a mean plasma ibuprofen Cₘₐₓ of about 15 mg/L to about 29 mg/L; (c) a mean plasma ibuprofen AUC_{0→12 h} of about 62 h·mg/L to about 77 h·mg/L; (d) a mean plasma ibuprofen AUC_{0→∞} of about 66·mg/L to about 79·mg/L; (e) a mean ibuprofen half-life (t½) of about 2.3 h to about 2.4 h; or (f) a mean ibuprofen terminal elimination rate constant (λ_{z}) of about 0.29 h⁻¹ to about 0.31 h⁻¹. In another aspect described herein, administering the composition provides one or more of the following pharmacokinetic parameters: (a) a mean plasma ibuprofen Tₘₐₓ of about 0.7 hours under fasting conditions; (b) a mean plasma ibuprofen Cₘₐₓ of about 29 mg/L under fasting conditions; (c) a mean plasma ibuprofen AUC_{0→12 h} of about 77 h·mg/L under fasting conditions; (d) a mean plasma ibuprofen AUC_{0→∞} of about 79·mg/L under fasting conditions; (e) a mean ibuprofen half-life (t½) of about 2.4 h under fasting conditions; or (f) a mean ibuprofen terminal elimination rate constant (λ_{z}) of about 0.29 h⁻¹ under fasting conditions; or (g) a mean plasma ibuprofen Tₘₐₓ of about 1.8 hours under fed conditions; (h) a mean plasma ibuprofen Cₘₐₓ of about 15 mg/L under fed conditions; (i) a mean plasma ibuprofen AUC_{0→12 h} of about 62 h·mg/L under fed conditions; (j) a mean plasma ibuprofen AUC_{0→∞} of about 66·mg/L under fed conditions; (k) a mean ibuprofen half-life (t½) of about 2.3 h under fed conditions; or (l) a mean ibuprofen terminal elimination rate constant (λ_{z}) of about 0.31 h⁻¹ under fed conditions.

In another embodiment described herein, the administering a pharmaceutical composition described herein is sufficient to achieve a reduction of pain, reduction of inflammation, or reduction of fever relative to baseline in the subject without substantially inducing one or more of abdominal pain, acid or sour stomach, belching, bloating, cloudy urine, decrease in amount of urine, decrease in urine output or decrease in urine-concentrating ability, diarrhea, difficulty having a bowel movement (stool), excess air or gas in stomach or intestines, full feeling, heartburn, indigestion, itching skin, nausea, noisy, rattling breathing, pain or discomfort in chest, upper stomach, or throat, pale skin, passing gas, rash with flat lesions or small raised lesions on the skin, shortness of breath, swelling of face, fingers, hands, feet, lower legs, or ankles, troubled breathing at rest, troubled breathing with exertion, unusual bleeding or bruising, unusual tiredness or weakness, vomiting, or weight gain.

### BRIEF DESCRIPTION OF THE DRAWINGS

FIG. 1: Dissolution profiles of ibuprofen sodium soft gel capsules compared to ibuprofen sodium film-coated tablets, ibuprofen free acid tablets, and ibuprofen free acid liquigels in a USP basket test.

FIG. 2: Mean plasma ibuprofen concentrations from ibuprofen sodium soft gel capsules under fasting conditions compared to ibuprofen sodium film coated tablets.

FIG. 3: Mean plasma ibuprofen concentrations under fed conditions compared to ibuprofen sodium film coated tablets.

### DETAILED DESCRIPTION

Described herein are oral pharmaceutical compositions of ibuprofen, pharmacologically active ibuprofen salts, or combinations thereof.

The oral pharmaceutical compositions described herein provide soft gel capsules comprising matrix fills of ibuprofen salts, such as ibuprofen sodium, ibuprofen potassium, or combinations thereof, and methods for preparation thereof. Also described herein are compositions and methods for manufacturing rapid release ibuprofen salts, or combinations thereof as soft capsules.

As used herein, the term “ibuprofen” refers to the nonsteroidal anti-inflammatory drug (R/S)-2-(4-(2-methylpropyl)phenyl) propanoic acid, i.e., (±)-2-(p-isobutylphenyl) propionic acid, or any pharmacologically active salts, salt hydrates, or derivatives thereof, and combinations or mixtures of any of the foregoing. In one embodiment described herein, “ibuprofen” or “ibuprofen sodium” refers to ibuprofen sodium dihydrate. A dose of about 256 mg ibuprofen sodium dihydrate (C₁₃H₁₇O₂Na.2H₂O; molecular weight: 264.29 g·mol⁻¹) provides an equivalent dose as about 200 mg ibuprofen free acid (C₁₃H₁₈O₂; molecular weight: 206.29 g·mol⁻¹).

The terms “active ingredient” or “active pharmaceutical ingredient” as used herein refer to a pharmaceutical agent, active ingredient, compound, or substance, compositions, or mixtures thereof, that provide a pharmacological, often beneficial, effect.

The terms “dosage” or “dose” as used herein denote any forms of the active ingredient formulation that contain an amount sufficient to produce a therapeutic effect with a single administration. The dosage form described herein is for oral administration. The preferred oral dosage forms described herein are soft gelatin capsules or “soft gels.”

The terms “active pharmaceutical ingredient load” or “drug load” as used herein refers to the quantity (mass) of the active pharmaceutical ingredient comprised in a single soft capsule fill.

The term “formulation” or “composition” as used herein refers to the drug in combination with pharmaceutically acceptable excipients. This term includes orally administrable formulations as well as formulations administrable by other means.

The term “titration” as used herein refers to the incremental increase in drug dosage to a level that provides the optimal therapeutic effect.

The terms “immediate release” or “rapid release,” as used herein refer to a composition that releases the majority of the active ingredient shortly after ingestion by a subject. Typically, the active ingredient begins release as soon as a portion of dosage form begins dissolution.

The term “controlled release” as used herein refers to a composition that does not immediately releases an active ingredient. “Controlled release” as used herein encompasses the terms “modified release,” “sustained release,” “extended release,” and “delayed release.”

The term “delayed release” as used herein refers to a composition that releases an active ingredient according to a desired profile after an extended period of time under physiological conditions or in an in vitro test. By “extended period” it is meant a continuous period of time of at least about 1 hour; about 2 hours; about 4 hours; about 6 hours; about 8 hours; about 10 hours; or about 12 hours.

The term “modified release” as used herein refers to a composition that releases an active ingredient at a slower rate than does an immediate release formulation under physiological conditions or in an in vitro test.

The term “sustained” release” as used herein refers to a composition that releases an active ingredient over an extended period of time, for example minutes, hours, or days, such that less than all the active ingredient is released initially. A sustained release rate may provide, for example, a release of a certain specified amount of a drug or active ingredient from a dosage form, over a certain period, under physiological conditions or in an in vitro test.

The term “extended release” as used herein refers to a composition that releases an active ingredient over an extended period, such as of at least about 1 hour; about 2 hours; about 4 hours; about 6 hours; about 8 hours; about 10 hours; about 12 hours; about 14 hours; about 16 hours; about 18 hours; about 20 hours about 24 hours; or even longer; specifically over a period of at least 18 hours under physiological conditions or in an in vitro assay.

The term “Cₘₐₓ” as used herein refers to the maximum observed blood (plasma, serum, or whole blood) concentration or the maximum blood concentration calculated or estimated from a concentration to time curve, and is expressed in units of mg/L or μg/mL, as applicable.

The term “Cₘᵢₙ” as used herein refers to the minimum observed blood (plasma, serum, or whole blood) concentration or the minimum blood concentration calculated or estimated from a concentration to time curve, and is expressed in units of mg/L or μg/mL, as applicable.

The term “C_{avg}” as used herein refers to the blood (plasma, serum, or whole blood) concentration of the drug within the dosing interval, is calculated as AUC/dosing interval, and is expressed in units of mg/L or μg/mL, as applicable.

The term “Tₘₐₓ” as used herein refers to the time after administration at which Cₘₐₓ occurs, and is expressed in units of hours (h) or minutes (min), as applicable.

The term “AUC_{0∞τ}” as used herein refers to area under the blood (plasma, serum, or whole blood) concentration versus time curve from time zero to time tau (τ) over a dosing interval at steady state, where tau is the length of the dosing interval, and is expressed in units of h·mg/L or h·μg/mL, as applicable. For example, the term AUC_{0→12} as used herein refers to the area under the concentration versus time curve from 0 to 12 hours.

The term “AUC_{0→∞}” or “AUC_{INF}” as used herein refers to the area under the blood (plasma, serum, or whole blood) concentration versus time curve from time 0 hours to infinity, and is expressed in units of h·mg/L or h·μg/mL, as applicable.

The term “AUC_{L}” as used herein refers to the area under the blood (plasma, serum, or whole blood) concentration versus time curve from time 0 hours to the last measurable concentration of a drug or active pharmaceutical ingredient in the blood, and is expressed in units of h·mg/L, or h·μg/mL, as applicable.

The term “half-life” or “t½” as used herein refers to the time required for the concentration of the drug or active pharmaceutical ingredient to reach half of its original value, and is expressed in units of time, such as hours or minutes, as applicable.

The term “terminal elimination rate constant” or “λ_{z}” or “K” or “Kₑ” refers to the rate at which a drug is removed from the plasma (or the body), and is expressed in units of per time, such as h⁻¹ or min⁻¹, as applicable.

The phrase “under fed conditions” as used herein refers to a subject that has consumed food shortly prior to ingesting or contemporaneously while ingesting the pharmaceutical composition.

The phrase “under fasting conditions” as used herein refers to a subject that has fasted or abstained from consuming food for at least 10 hours prior to ingesting the pharmaceutical composition.

The term “treating” refers to administering a therapy in an amount, manner, or mode effective to improve a condition, symptom, or parameter associated with a disorder.

The term “prophylaxis” refers to preventing or reducing the progression of a disorder, either to a statistically significant degree or to a degree detectable to one skilled in the art.

The term “substantially” as used herein means to a great or significant extent, but not completely.

The term “about” as used herein refers to any value that is within a variation of up to ±10% of the value modified by the term “about.”

As used herein, “a” or “an” means one or more unless otherwise specified.

Terms such as “include,” “including,” “contain,” “containing” and the like mean “comprising.”

The term “or” can be conjunctive or disjunctive.

One embodiment described herein, is an oral immediate-release pharmaceutical composition comprising a soft capsule shell encapsulating a matrix fill comprising an ionic form of ibuprofen.

Another embodiment described herein is an oral immediate-release pharmaceutical composition comprising a soft capsule and encapsulated matrix comprising an active pharmaceutical ingredient. In one embodiment, the immediate release oral pharmaceutical composition comprises one or more NSAID active pharmaceutical ingredients. In another embodiment, the immediate release oral pharmaceutical composition comprises one or more NSAID active ingredients in a liquid formulation. In another embodiment, the one or more NSAID active pharmaceutical ingredients are ionic salt forms.

One embodiment described herein is oral immediate-release pharmaceutical composition comprising a soft capsule encapsulating a liquid matrix comprising one or more NSAID active pharmaceutical ingredients in a hydrophilic vehicle as shown in Table 1.

TABLE 1
Exemplary Matrix Fill Composition

| Exemplary Ingredients | mg/capsule | % weight |
|---|---|---|
| Hydrophilic vehicle (e.g., one or | 300-580 | 30-70 |
| more polyethylene glycols) | | |
| Co-solvent (e.g., propylene glycol, glycerol) | 30-150 | 3-5 |
| pH modifier (e.g., lactate, citrate, etc) | 30-80 | 3-10 |
| NSAID (e.g., ibuprofen, naproxen, | 200-800 | 20-40 |
| diclofenac, etc.) | | |
| TOTAL | 800-1200 | 100 |

In another embodiment, the hydrophilic vehicle comprises one or more hydrophilic vehicles. Suitable hydrophilic vehicles or solvents described herein are anhydrous and compatible with soft gelatin capsules. Non-limiting exemplary vehicles comprise Capmul® MCM, Captex® 355, Cremophor® RH 40, Croscarmellose, Crospovidone, Crospovidone CL, Crospovidone CL-F, Crospovidone CL-M, Imwitor® 742, Kollidon® CL, Kollidon® CL-F, Kollidon® CL-M, Labrafac™ Lipophile WL 1349, Labrafil® M2125CS, Labrasol®, Lutrol® F 68, Maisine™ 35-1, mannitol, Miglyol® 812, Pearlitol® Flash, Peceol®, Plural® Oleique CC 497, Povidone K 17, Povidone K 30, polyethylene glycol 200, polyethylene glycol 400, polyethylene glycol 600, polyethylene glycol 800, polyethylene glycol 1000, polyethylene glycol 2000, polyethylene glycol 3350, propylene glycol, glycerol, or mixtures thereof. In on embodiment the hydrophilic vehicle comprises one or more hydro-alcohols including polyethylene glycols of a molecular weight ranging from about 200 to about 8000 or a mixture or combination thereof.

In another embodiment, the hydrophilic vehicle may comprise one or more disintegrant excipients. Disintegrants comprise any polymer, which expands in aqueous solution causing a tablet or capsule to burst and facilitate dissolution. Exemplary, non-limiting disintegrants comprise crosslinked polyvinylpyrrolidone (e.g., crospovidone), crosslinked sodium carboxymethyl cellulose (croscarmellose sodium) carboxymethyl cellulose calcium, cysteine HCl, modified starches (e.g., sodium starch glycolate), cellulose, calcium silicate, silicon dioxide, alginic acid, sodium alginate, citric acid, microcrystalline cellulose, polyoxy stearate, sodium carmellose, sodium lauryl sulfate, or a mixture or combination thereof.

In another embodiment, the hydrophilic vehicle may comprise one or more surfactants. The surfactant can have a hydrophilic/lipophilic balance (HLB) value between about 1 and about 25 and a melting point between about 25° C. and about 70° C. The HLB characteristic of surfactants can be determined in accordance with “Physical Pharmacy: Physical Chemical Principles in the Pharmaceutical Sciences,” Fourth Edition, pp. 371-373, A. Martin, Ed., Lippincott Williams & Wilkins, Philadelphia (1993). Suitable, non-limiting surfactants include: glyceryl monocaprylate (e.g., Capmul® MCM), Pluronic® 10R5, Pluronic® 17R2, Pluronic® 17R4, Pluronic® 25R2, Pluronic® 25R4, Pluronic® 31R1, Pluronic® F 108, Pluronic® F 108 NF, Pluronic® F 108, Pluronic® F 108NF, Poloxamer 338, Pluronic® F 127, Pluronic® F 127 NF, Pluronic® F 127 NF 500 BHT Prill, Pluronic® F 127 NF Prill, Poloxamer 407, Pluronic® F 38, Pluronic® F 38 Pastille, Pluronic® F 68, Pluronic® F 68 LF Pastille, Pluronic® F 68 NF, Pluronic® F 68 NF Prill, Poloxamer 188, Pluronic® F 68 Pastille, Pluronic® F 77, Pluronic® F 77 Micropastille, Pluronic® F 87, Pluronic® F 87 NF, Pluronic® F 87 NF Prill, Poloxamer 237, Pluronic® F 88, Pluronic® F 88 Pastille, Pluronic® F 98, Pluronic® L 10, Pluronic® L 101, Pluronic® L 121, Pluronic® L 31, Pluronic® L 35, Pluronic® L 43, Pluronic® L 61, Pluronic® L 62, Pluronic® L 62 LF, Pluronic® L 62D, Pluronic® L 64, Pluronic® L 81, Pluronic® L 92, Pluronic® N 3, Pluronic® P 103, Pluronic® P 104, Pluronic® P 105, Pluronic® P 123 Surfactant, Pluronic® P 65, Pluronic® P 84, Pluronic® P 85, Adogen® 464, Alkanol® 6112, Brij® 52, Brij® 93, Brij® S2, Brij® S, Brij® 58, Brij® C10, Brij® L4, Brij® O10, Brij® O10, BRIJ® O20, Brij® S10, Brij® S20, ethylenediamine tetrakis(ethoxylate-block-propoxylate) tetrol, ethylenediamine tetrakis(ethoxylate-block-propoxylate) tetrol, ethylenediamine tetrakis(propoxylate-block-ethoxylate) tetrol, IGEPAL® CA-210, IGEPAL® CA-520, IGEPAL® CA-720, IGEPAL® CO-520, IGEPAL® CO-630, IGEPAL® CO-720, IGEPAL® CO-890, IGEPAL® DM-970, MERPOL® DA, MERPOL® HCS, MERPOL® OJ, MERPOL® SE, MERPOL® SH, MERPOL® A, Poly(ethylene glycol) sorbitan tetraoleate, poly(ethylene glycol) sorbitol hexaoleate, poly(ethylene glycol) (12), poly(ethylene glycol) (18), polyethylene-block-poly(ethylene glycol), sorbitan monopalmitate, 2,4,7,9-tetramethyl-5-decyne-4,7-diol ethoxylate, Nonidet™ P-40, Triton™ N-101, Triton™ X-100, Triton™ X-114, Triton™ X-405, TWEEN® 20, TWEEN® 40, TWEEN® 60, TWEEN® 85, Zonyl® FS-300, or Zonyl® FSN or a mixture or combination thereof.

In another embodiment, the hydrophilic vehicle may comprise a hygroscopic polymer. In one embodiment, the hygroscopic polymers include polyvinylpyrrolidone, copovidone, hydroxypropylmethylcellulose, hydroxypropylcellulose, ethyl cellulose, methylcellulose, and polyethylene oxide. Suitable hygroscopic polymers include polyvinyl alcohol, a copolymer of polyvinylpyrrolidone and polyvinyl acetate, hydroxypropyl cellulose, hydroxypropyl methylcellulose, hydroxyethyl cellulose, hydroxymethyl cellulose, gelatin, polyethylene oxide, such as POLYOX™ 100,000-600,000 MW, acacia, dextrin, starch, polyhydroxyethylmethacrylate, a water-soluble non-ionic polymethacrylate or copolymer thereof, a modified cellulose, a modified polysaccharide, a non-ionic gum, or a non-ionic polysaccharide.

In another embodiment, the hydrophilic vehicle may comprise one or more lipids or lipophilic vehicles. In one aspect, the lipid or lipophilic vehicle may be a liquid or a solid or a semisolid lipid or lipophilic vehicle. Suitable non-limiting liquid lipid or lipophilic vehicles comprise olive oil, soybean oil, sunflower oil, canola oil, palmitoleic acid, oleic acid, myristoleic acid, linoleic acid, arachidonic acid, paraffin oil, or mineral oil or a mixture or combination thereof. The lipid or lipophilic vehicle can be a semi-solid lipophilic vehicle such as a polyethylene glycol glyceride ester, e.g., Gelucire® 33/01, Gelucire® 37/02, Gelucire® 39/01, Gelucire® 43/01, Gelucire® 44/14, Gelucire® 50/02, Gelucire® 50/13, Gelucire® 53/10, or Gelucire® 62/02; a paraffin wax, carnauba wax, or bee's wax.

In another embodiment, the hydrophilic vehicle may comprise one or more pH modifying agents or neutralizing agents. Suitable non-limiting examples of such agents include acetic acid, ammonium carbonate, ammonium phosphate, boric acid, carbonic acid, citric acid, dibasic sodium phosphate, diluted hydrochloric acid, diluted phosphoric acid, fumaric acid, glacial acetic acid, hydrochloric acid, lactic acid, malic acid, monobasic sodium phosphate, nitric acid, phosphoric acid, potassium citrate, potassium metaphosphate, potassium phosphate monobasic, sodium acetate, sodium citrate, sodium lactate solution, sulfuric acid, tartaric acid, sodium hydroxide, ammonium hydroxide, potassium hydroxide, sodium bicarbonate, sodium carbonate, or a mixture or combination thereof.

Additional pharmaceutical excipients useful for the pharmaceutical composition described herein include, for example, the following: Alkalizing agents (ammonia solution, ammonium carbonate, diethanolamine, diisopropanolamine, potassium hydroxide, sodium bicarbonate, sodium borate, sodium carbonate, sodium hydroxide, trolamine); Antifoaming agents (dimethicone, simethicone); Antimicrobial preservatives (benzalkonium chloride, benzalkonium chloride solution, benzethonium chloride, benzoic acid, benzyl alcohol, butylparaben, cetylpyridinium chloride, chlorobutanol, chlorocresol, cresol, dehydroacetic acid, ethylparaben, methylparaben, methylparaben sodium, phenol, phenylethyl alcohol, phenylmercuric acetate, phenylmercuric nitrate, potassium benzoate, potassium sorbate, propylparaben, propylparaben sodium, sodium benzoate, sodium dehydroacetate, sodium propionate, sorbic acid, thimerosal, thymol); Antioxidants (ascorbic acid, ascorbyl palmitate, butylated hydroxyanisole, butylated hydroxytoluene, hypophosphorous acid, monothioglycerol, propyl gallate, sodium formaldehyde sulfoxylate, sodium metabisulfite, sodium thiosulfate, sulfur dioxide, tocopherol, tocopherols excipient); Chelating agents (edetate disodium, ethylenediaminetetraacetic acid and salts, edetic acid); Coating agents (sodium carboxymethylcellulose, cellulose acetate, cellulose acetate phthalate, ethylcellulose, gelatin, pharmaceutical glaze, hydroxypropyl cellulose, hydroxypropyl methylcellulose, hydroxypropyl methylcellulose phthalate, methacrylic acid copolymer, methylcellulose, polyvinyl acetate phthalate, shellac, sucrose, titanium dioxide, carnauba wax, microcrystalline wax, zein); Colorants (caramel, red, yellow, black or blends, ferric oxide); Complexing agents (ethylenediaminetetraacetic acid and salts (EDTA), edetic acid, gentisic acid ethanolamide, oxyquinoline sulfate); Desiccants (calcium chloride, calcium sulfate, silicon dioxide); a Wetting agent, such as lecithin; Emulsifying and/or solubilizing agents (acacia, cholesterol, diethanolamine (adjunct), glyceryl monostearate, lanolin alcohols, mono- and di-glycerides, monoethanolamine (adjunct), oleic acid (adjunct), oleyl alcohol (stabilizer), poloxamer, polyoxyethylene 50 stearate, polyoxyl 35 castor oil, polyoxyl 40 hydrogenated castor oil, polyoxyl 10 oleyl ether, polyoxyl 20 cetostearyl ether, polyoxyl 40 stearate, polysorbate 20, polysorbate 40, polysorbate 60, polysorbate 80, diacetate, monostearate, sodium lauryl sulfate, sodium stearate, sorbitan monolaurate, sorbitan monooleate, sorbitan monopalmitate, sorbitan monostearate, stearic acid, trolamine, emulsifying wax); Filtering aids (powdered cellulose, purified siliceous earth); Flavors and perfumes (anethole, benzaldehyde, ethyl vanillin, menthol, methyl salicylate, monosodium glutamate, orange flower oil, peppermint, peppermint oil, peppermint spirit, rose oil, stronger rose water, thymol, tolu balsam tincture, vanilla, vanilla tincture, vanillin); Humectants (glycerin, hexylene glycol, sorbitol); Plasticizers (e.g., castor oil, diacetylated monoglycerides, diethyl phthalate, glycerin, mono- and di-acetylated monoglycerides, propylene glycol, triacetin, triethyl citrate); polymers (e.g., cellulose acetate, alkyl celluloses, hydroxyalkyl, acrylic polymers and copolymers); Solvents (acetone, alcohol, diluted alcohol, amylene hydrate, benzyl benzoate, butyl alcohol, carbon tetrachloride, chloroform, corn oil, cottonseed oil, ethyl acetate, glycerin, hexylene glycol, isopropyl alcohol, methyl alcohol, methylene chloride, methyl isobutyl ketone, mineral oil, peanut oil, propylene carbonate, sesame oil, water for injection, sterile water for injection, sterile water for irrigation, purified water); Sorbents (powdered cellulose, charcoal, purified siliceous earth); Carbon dioxide sorbents (barium hydroxide lime, soda lime); Stiffening agents (hydrogenated castor oil, cetostearyl alcohol, cetyl alcohol, cetyl esters wax, hard fat, paraffin, polyethylene excipient, stearyl alcohol, emulsifying wax, white wax, yellow wax); Suspending and/or viscosity-increasing agents (acacia, agar, alginic acid, aluminum monostearate, bentonite, purified bentonite, magma bentonite, carbomer, carboxymethylcellulose calcium, carboxymethylcellulose sodium, carboxymethylcellulose sodium 12, carrageenan, microcrystalline and carboxymethylcellulose sodium cellulose, dextrin, gelatin, guar gum, hydroxyethyl cellulose, hydroxypropyl cellulose, hydroxypropyl methylcellulose, magnesium aluminum silicate, methylcellulose, pectin, polyethylene oxide, polyvinyl alcohol, povidone, alginate, silicon dioxide, colloidal silicon dioxide, sodium alginate, tragacanth, xanthan gum); Sweetening agents (aspartame, dextrates, dextrose, excipient dextrose, fructose, mannitol, saccharin, calcium saccharin, sodium saccharin, sorbitol, solution sorbitol, sucrose, compressible sugar, confectioner's sugar, syrup); binders (acacia, alginic acid, sodium carboxymethylcellulose, microcrystalline cellulose, dextrin, ethylcellulose, gelatin, liquid glucose, guar gum, hydroxypropyl methylcellulose, methylcellulose, polyethylene oxide, povidone, pregelatinized starch, syrup); capsule diluents (calcium carbonate, dibasic calcium phosphate, tribasic calcium phosphate, calcium sulfate, microcrystalline cellulose, powdered cellulose, dextrates, dextrin, dextrose excipient, fructose, kaolin, lactose, mannitol, sorbitol, starch, pregelatinized starch, sucrose, compressible sugar, confectioner's sugar); capsule lubricants (calcium stearate, glyceryl behenate, magnesium stearate, light mineral oil, sodium stearyl fumarate, stearic acid, purified stearic acid, talc, hydrogenated vegetable oil, zinc stearate); Tonicity agent (dextrose, glycerin, mannitol, potassium chloride, sodium chloride); Vehicle: flavored and/or sweetened (aromatic elixir, compound benzaldehyde elixir, iso-alcoholic elixir, peppermint water, sorbitol solution, syrup, tolu balsam syrup); Vehicle: oleaginous (almond oil, corn oil, cottonseed oil, ethyl oleate, isopropyl myristate, isopropyl palmitate, mineral oil, light mineral oil, myristyl alcohol, octyl dodecanol, olive oil, peanut oil, persic oil, sesame oil, soybean oil, squalane); Vehicle: solid carrier (sugar spheres); Vehicle: sterile (Bacteriostatic water for injection, bacteriostatic sodium chloride injection); Viscosity-increasing (see suspending agent); Water repelling agent (cyclomethicone, dimethicone, simethicone); and/or solubilizing agent (benzalkonium chloride, benzethonium chloride, cetylpyridinium chloride, docusate sodium, nonoxynol 9, nonoxynol 10, octoxynol 9, poloxamer, polyoxyl 35 castor oil, polyoxyl 40, hydrogenated castor oil, polyoxyl 50 stearate, polyoxyl 10 oleyl ether, polyoxyl 20, cetostearyl ether, polyoxyl 40 stearate, polysorbate 20, polysorbate 40, polysorbate 60, polysorbate 80, sodium lauryl sulfate, sorbitan monolaurate, sorbitan monooleate, sorbitan monopalmitate, sorbitan monostearate, tyloxapol). This non-limiting list is merely representative of the classes of excipients and the particular excipients that may be used in the pharmaceutical compositions as described herein.

Suitable hydrophilic fills for solubilizing active pharmaceutical ingredients are described in International Patent Application Publication No. WO 2006/096580, U.S. Patent Application Publication No. US 2007/0053868, and U.S. Pat. No. 8,333,989, each of which is incorporated by reference herein for such teachings.

In one embodiment described herein, the oral immediate-release pharmaceutical composition comprises a matrix fill comprising a hydrophilic vehicle. In one embodiment, the hydrophilic vehicle comprises one or more hydrophilic solvents. In one aspect, the hydrophilic vehicle comprises one or more polyethylene glycols. In one aspect, the hydrophilic vehicle comprises one or more polyethylene glycols having molecular weights of 200 to 3350, including, but not limited to 200, 400, 600, 800, 1000, 2000, and 3350. As used herein, low molecular weight polyethylene glycols have a molecular weight range of less than about 500. As used herein, high molecular weight polyethylene glycols have a molecular weight range greater than or equal to about 600. In one aspect, the hydrophilic vehicle comprises a low molecular weight polyethylene glycol. In another aspect, the hydrophilic vehicle comprises a high molecular weight polyethylene glycol. In another aspect, the hydrophilic vehicle comprises a mixture of a high molecular weight polyethylene glycol and a low molecular weight polyethylene glycol. In one aspect, the polyethylene glycol (PEG) is PEG 400. In another aspect, the PEG is PEG 600. In another aspect, the PEG is a mixture of PEG 400 and PEG 600.

In another embodiment described herein, the matrix fill comprises one or more hydrophilic co-solvents. In one embodiment, the hydrophilic co-solvent comprises one or more of glycerol, propylene glycol, polyethylene glycol, triethylene glycol, ethanol, or combinations thereof. In one aspect, the hydrophilic co-solvent comprises propylene glycol.

In another embodiment described herein, the matrix fill comprises one or more pH modifiers. In one embodiment, the pH modifier comprises an organic acid. In one embodiment, the pH modifier comprises one or more of citric acid, lactic acid, malic acid, tartaric acid, pyruvic acid, acetic acid, or combinations thereof. In one embodiment, the pH modifier comprises lactic acid.

In one embodiment, the hydrophilic vehicle comprises about 30% to about 70% by weight of the matrix fill, including each integer within the specified range. In one aspect, the total hydrophilic vehicle comprises about 30%, about 35%, about 40%, about 45%, about 50%, about 55%, about 60%, about 65%, or about 70% by weight of the matrix fill.

In one embodiment, PEG 400 comprises about 5% to about 50% by weight of the matrix fill, including each integer within the specified range. In one aspect, PEG 400 comprises about 5%, about 10%, about 15%, about 20%, about 25%, about 30%, about 35%, about 40%, about 45%, or about 50% by weight of the matrix fill.

In one embodiment, PEG 600 comprises about 25% to about 70% by weight of the matrix fill, including each integer within the specified range. In one aspect, PEG 600 comprises about 20%, about 25%, about 30%, about 35%, about 40%, about 45%, about 50%, about 55%, about 60%, about 65%, or about 70% by weight of the matrix fill.

In one embodiment, the ratio of PEG 600 to PEG 400 in the matrix fill comprises about 14:1 to about 1:2, including each ratio within the specified range. In one aspect, the ratio of PEG 600 to PEG 400 in the matrix fill comprises about 14:1, about 8:3, about 2:1, about 1:1, about 7:8, or about 1:2. In one aspect, the ratio of about PEG 600 to PEG 400 in the matrix fill comprises about 2:1.

In one embodiment, the co-solvent comprises about 2% to about 8% by weight of the matrix fill, including each integer within the specified range. In one aspect, the co-solvent comprises about 2.0%, about 2.5%, about 3%, about 3.5%, about 4%, about 4.5%, about 5%, or about 5.5%, by weight of the matrix fill.

In one embodiment, the ratio of hydrophilic vehicle (e.g., total PEG content) to cosolvent in the matrix fill comprises about 25:1 to about 5:1, including each ratio within the specified range. In one aspect, the ratio of PEG to cosolvent in the matrix fill comprises about 25:1, about 20:1, about 15:1, about 10:1, about 7.5:1, or about 5:1. In one aspect, the ratio of PEG to cosolvent in the matrix fill comprises about 14:1. In another aspect, the ratio of PEG to cosolvent in the matrix fill comprises about 20:1.

In one embodiment, the pH modifier comprises about 3% to about 10% by weight of the matrix fill, including each integer within the specified range. In one aspect, the co-solvent comprises about 2.5%, about 3%, about 3.5%, about 4%, about 4.5%, about 5%, or about 5.5%, about 6%, about 6.5%, about 7%, about 7.5%, about 8%, about 8.5%, about 9%, about 9.5%, about 10%, or about 10.5%, by weight of the matrix fill.

In one embodiment, the ratio of total hydrophilic vehicle (e.g., the combined weight percentage of the hydrophilic vehicle and cosolvent) to the pH modifier in the matrix fill comprises about 20:1 to about 9:1, including each ratio within the specified range. In one aspect, the ratio of total hydrophilic vehicle to the pH modifier in the matrix fill comprises of about 20:1, about 19:1, about 15:1, about 10:1, about 9:1, or about 8:1. In one aspect, the ratio of total hydrophilic vehicle to the pH modifier in the matrix fill comprises about 11:1. In another aspect, the ratio of total hydrophilic vehicle to the pH modifier in the matrix fill comprises about 8:1.

In one embodiment, the active pharmaceutical agent comprises about 20% to about 40% by weight of the matrix fill, including each integer within the specified range. In one aspect, the active pharmaceutical agent comprises about 15%, about 20%, about 25%, about 30%, about 35%, or about 40% by weight of the matrix fill.

In one embodiment, the ratio of active pharmaceutical agent to the total hydrophilic vehicle (e.g., the combined weight percentage of the hydrophilic vehicle and cosolvent) comprises about 1:1.4 to about 1:8.5, including each ratio within the specified range. In another embodiment, the ratio of active pharmaceutical agent to the total hydrophilic vehicle comprises about 1:2 to about 1:4, including each ratio within the specified range. In another embodiment, the ratio of active pharmaceutical agent to the total hydrophilic vehicle comprises about 1:2 to about 1:2.5, including each ratio within the specified range. In one aspect, the ratio of active pharmaceutical agent to the total hydrophilic vehicle (e.g., hydrophilic vehicle and cosolvent) comprises about 1:1.5, about 1:2, about 1:2.1, about 1:2.2, about 1:2.3, about 1:2.4, about 1:2.5, about 1:3, about 1:4, about 1:6 or about 1:8.4. In one aspect, the ratio of active pharmaceutical agent to the total hydrophilic vehicle comprises about 1:2. In another aspect, the ratio of active pharmaceutical agent to the total hydrophilic vehicle comprises about 1:2.1. In another aspect, the ratio of active pharmaceutical agent to the total hydrophilic vehicle comprises about 1:2.2. In another aspect, the whole number ratio of active pharmaceutical agent to the total hydrophilic vehicle comprises about 0.48. In another aspect, the whole number ratio of active pharmaceutical agent to the total hydrophilic vehicle comprises about 0.45.

In one embodiment, the ratio of active pharmaceutical agent to the pH modifier in the matrix fill comprises about 1:1 to about 8:1, including each ratio within the specified range. In one embodiment the ratio of active pharmaceutical agent to the pH modifier in the matrix fill comprises about 3.5:1 to about 5.5:1, including each ratio within the specified range. In one embodiment the ratio of active pharmaceutical agent to the pH modifier in the matrix fill comprises about 4:1 to about 5:1, including each ratio within the specified range. In one embodiment, the ratio of active pharmaceutical agent to the pH modifier in the matrix fill comprises about 1:1, about 1.7:1, about 2.5:1, about 3.5:1, about 4:1, about 4.25:1, about 4.5:1, about 4.75:1, about 5:1, about 5.5:1, about 6:1, or about 8:1. In one aspect, the ratio of active pharmaceutical agent to the pH modifier comprises about 3.7:1. In another aspect, the ratio of active pharmaceutical agent to the pH modifier comprises about 4.8:1.

In another embodiment described herein, the total mass of the matrix fill of the pharmaceutical composition described herein that comprises an active pharmaceutical ingredient described herein is from about 300 mg to about 1500 mg, including all integers within the specified range. In one aspect, the total mass of the matrix fill mass is about 300 mg. In another aspect, the total mass of the matrix fill mass is about 400 mg. In one aspect, the total mass of the matrix fill mass is about 500 mg. In another aspect, the total mass of the matrix fill mass is about 600 mg. In another aspect, the total mass of the matrix fill mass is about 700 mg. In another aspect, the total mass of the matrix fill mass is about 800 mg. In another aspect, the total mass of the matrix fill mass is about 800 mg. In another aspect, the total mass of the matrix fill mass is about 900 mg. In another aspect, the total mass of the matrix fill mass is about 1000 mg. In another aspect, the total mass of the matrix fill mass is about 1100 mg. In another aspect, the total mass of the matrix fill mass is about 1200 mg. In another aspect, the total mass of the matrix fill mass is about 1300 mg. In another aspect, the total mass of the matrix fill mass is about 1400 mg. In another aspect, the total mass of the matrix fill mass is about 1500 mg.

In one embodiment described herein, the ratio of the active pharmaceutical ingredient to the matrix vehicle (e.g., the matrix components other than the active pharmaceutical ingredient comprising the combined weight percentage of the hydrophilic vehicle, cosolvent, and pH modifier) is about 1:1.5 to about 1:9, including each ratio within the specified range. In another embodiment, the ratio of active pharmaceutical agent to the matrix vehicle comprises about 1:2 to about 1:3, including each ratio within the specified range. In another embodiment, the ratio of active pharmaceutical agent to the matrix vehicle comprises about 1:1.5, about 1:2, about 1:2.4, about 1:3, about 1:4, about 1:5, about 1:6, about 1:7, about 1:8, or about 1:9. In one aspect, the ratio of the active pharmaceutical ingredient to the matrix vehicle is about 1:2.4. In another aspect, the weight ratio of the active pharmaceutical ingredient to the matrix vehicle is about 1:2.5.

In one embodiment described herein, the pharmaceutical composition comprises a matrix fill comprising about 30-70% of at least one hydrophilic vehicle; about 3-5% of at least one co-solvent; about 3-10% of at least one pH modifier; and about 20-40% of one or more active pharmaceutical ingredients. In one aspect, the pharmaceutical composition comprises a matrix fill comprising one or more polyethylene glycols, propylene glycol, lactic acid, and a salt form of ibuprofen.

In one embodiment described herein, the pharmaceutical composition comprises a matrix fill comprising about 39% polyethylene glycol 600; about 23% polyethylene glycol 400; about 3% propylene glycol; about 6% lactic acid; and about 30% ibuprofen, sodium salt.

In one embodiment described herein, the pharmaceutical composition comprises a matrix fill comprising about 500 mg polyethylene glycol 600; about 34 mg propylene glycol; about 53 g lactic acid; and about 260 mg ibuprofen, sodium salt.

In one embodiment described herein, the pharmaceutical composition comprises a matrix fill comprising about 345 mg polyethylene glycol 600; about 200 mg polyethylene glycol 400; about 26 mg propylene glycol; about 69 g lactic acid; and about 260 mg ibuprofen, sodium salt.

In one embodiment described herein, the pharmaceutical composition described herein comprises a soft capsule shell and a matrix fill. In one embodiment, the soft capsule shell has the composition of Table 2, including all possible iterations of the specified ranges that provide 100% for the total weight percentage, including or excluding the optional colorings, flavorings, or excipients.

TABLE 2
Exemplary soft gelatin capsule composition

| | Exemplary | Weight |
|---|---|---|
| Component | Component | Percentage (%) |
| Film-forming polymer | Gelatin | 25-50 |
| Plasticizer | Glycerol, sorbitol, | 15-25 |
| | combinations | |
| Solvent | Water | 20-40 |
| Opacifier (optional) | Titanium dioxide | 0.5-1.5 |
| Coloring agent (optional) | Various | 0.05-0.1 |
| TOTAL | | 100% |

Film-former polymers that are useful for creating soft capsules as described herein are gelatin or hydroxypropylmethylcellulose (HPMC). In one aspect, the film-forming polymer is gelatin.

Plasticizers that are useful for creating soft capsules as described herein are glycerol, sorbitol, polyethylene glycols, or combinations thereof. The weight ratio between the film-forming polymer, plasticizer, and solvent is adjusted so that the gel mass is flowable and not too viscous, and can be made into soft capsules using rotary die encapsulation methods.

In one embodiment, the enteric soft capsule shell has the exemplary composition shown in Table 3.

TABLE 3
Exemplary Soft Capsule Shell Composition

| Component | Percent weight (%) |
|---|---|
| Gelatin | 43 |
| Glycerol | 20 |
| Titanium dioxide (optional) | 0.7 |
| Coloring agent (optional) | 0.1 |
| Water | 36.2 |
| TOTAL | 100% |
| Final pH | ~4-7 |
| Ratio total plasticizer to gelatin | 20:43 (0.46:1) |
| Water content in dried soft capsule shell: | 8-15% |

In one embodiment described herein, the soft capsule comprises about 43% of at least one film-forming polymer; about 20% of at least one plasticizer; about 36% water; optionally, about 0.7% titanium dioxide; and optionally, about 0.1% of at least one coloring agent.

In one embodiment, the weight percentage range of film-forming polymer of the soft capsule described herein is about 35% to about 45%, including all integers within the specified range. In one aspect, the film-forming polymer weight percentage is about 38%. In another aspect, the film-forming|polymer weight percentage is about 42%. In another aspect, the film-forming polymer weight percentage is about 44%.

In one embodiment, the weight percentage range of plasticizer is about 15% to about 22%, including all iterations of integers with the specified range. In one aspect, the plasticizer weight percentage is about 17%. In another aspect, the plasticizer weight percentage is about 18.5%. In another aspect, the plasticizer weight percentage is about 20%.

In one embodiment, the weight percentage ratio range of plasticizer to film-forming polymer is about 0.33:1 to about 0.56:1, including all iterations of iterations of ratios with the specified range. In one embodiment, the weight percentage ratio range of plasticizer to film-forming polymer is about 0.38:1. In one embodiment, the weight percentage ratio range of plasticizer to film-forming polymer is about 0.42:1. In one embodiment, the weight percentage ratio range of plasticizer to film-forming polymer is about 0.46:1. In one embodiment, the weight percentage ratio range of plasticizer to film-forming polymer is about 0.52:1.

In one embodiment, soft capsules are made using a rotary die apparatus as described in U.S. Pat. Nos. 5,459,983; 5,146,730; and 6,482,516, each of which are incorporated by reference herein for such teachings.

Another embodiment described herein includes a process of manufacturing soft capsules comprising the pharmaceutical composition as described herein. The process includes preparing a gel mass composition comprising a film-forming, water-soluble polymer, an appropriate plasticizer, and solvent; casting the gel mass into films or ribbons using heat-controlled drums or surfaces; and manufacturing a soft capsule comprising a matrix fill using rotary die technology. The thickness of the films or ribbons that form the soft capsule shell is from about 0.010 inches (≈0.254 mm) to about 0.050 inches (≈1.27 mm), including all integers within the specified range. The shell thickness can be about 0.010 inch (≈0.254 mm), about 0.015 inch (≈0.381 mm), about 0.02 in (≈0.508 mm), about 0.03 in (≈0.762 mm), about 0.04 in (≈1.02 mm), or about 0.05 in (≈1.27 mm). In one embodiment, the thickness is about 0.02 inches (≈0.508 mm) to about 0.040 inches (≈1.02 mm). In one embodiment, the shell thickness is about 0.028 inches (≈0.711 mm). In another embodiment, the shell thickness is about 0.033 inches (≈0.838 mm). In another embodiment, the shell thickness is about 0.038 inches (≈0.965 mm).

In one embodiment described herein, the soft capsule shell described herein, encapsulates a matrix fill as described herein. In another embodiment described herein, the soft capsule shell and encapsulated matrix fill comprises a outer dimension from about 2 oval to about 30 oval including all iterations of capsule size within the specified range (e.g., 2 oval, 3 oval, 4 oval, 5 oval, 6 oval, 7 oval, 8 oval, 10 oval, 12 oval, 16 oval, 20, or 30 oval). In another embodiment described herein, the soft capsule shell and encapsulated matrix fill comprises a outer dimension from about 2 round to about 28 round including all iterations of capsule size within the specified range (e.g., 2 round, 3 round, 4 round, 5 round, 6 round, 7 round, 8 round, 10 round, 12 round, 16 round, 20 round or 28 round). In another embodiment described herein, the soft capsule shell and encapsulated matrix fill comprises a outer dimension from about 2 oblong to about 22 oblong including all iterations of capsule size within the specified range (e.g., 2 oblong, 3 oblong, 4 oblong, 5 oblong, 6 oblong, 7 oblong, 8 oblong, 10 oblong, 11, oblong, 12 oblong, 14 oblong, 16 oblong, 20 oblong, or 22 oblong). Dimension specifications of soft capsules and tablets are known to those skilled in the art. See Remington's Essentials of Pharmaceutics, Pharmaceutical Press Publishing Company, London, UK, 1^{st} Edition, 2013, which is incorporated by reference herein for such teachings.

In one embodiment described herein, the soft capsules described herein comprise an active pharmaceutical ingredient. In one embodiment described herein, an active pharmaceutical ingredient is the only active ingredient in the pharmaceutical composition. In another embodiment, one or more active ingredients or drugs are included in the pharmaceutical composition. In another embodiment, the active ingredient or drug can be an active pharmaceutical ingredient, derivatives thereof, or combinations thereof.

In one embodiment described herein, the active pharmaceutical ingredient is one or more non-steroidal anti-inflammatory drugs (NSAID). Non-limiting examples of NSAID active pharmaceutical ingredients comprise aspirin, ibuprofen, aceclofenac, acemetacin, aloxiprin, azapropazone, benorilate, bromfenac, carprofen, celecoxib, choline magnesium salicylate, diclofenac, diflunisal, etodolac, etoricoxib, faislamine, fenbufen, fenoprofen, flurbiprofen, indometacin, ketoprofen, ketorolac, lornoxicam, loxoprofen, meloxicam, meclofenamic acid, mefenamic acid, meloxicam, metamizole, methyl salicylate, magnesium salicylate, nabumetone, naproxen, nimesulide, oxyphenbutazone, parecoxib, phenylbutazone, piroxicam, salicyl salicylate, sulindac, sulfinpyrazone, suprofen, tenoxicam, tiaprofenic acid, tolmetin, or valdecoxib.

Without being bound to any theory, ibuprofen's and other NSAID's anti-inflammatory, antipyretic, and analgesic actions are believed to result from the inhibition of cyclooxygenase-2 (COX-2). COX-2 is required to convert arachidonic acid to prostaglandin H2 (PGH2). The inhibition of COX-2 by ibuprofen therefore lowers the level of prostaglandins made by the body.

In another embodiment, the active pharmaceutical ingredient is one or more NSAIDs combined with one or more cold, cough, allergy, nasal decongestant, antitussive, expectorant, antihistamine, stimulant, sedative, anti-inflammatory, antibiotic, anti-viral, anti-asthmatic, anti-migraine, hypnotic, narcotic analgesic, or narcotic antagonist active pharmaceutical ingredients, or further combinations thereof.

In another embodiment, the active pharmaceutical ingredient can comprise one or more NSAIDs combined with one or more nasal decongestants, antitussives, expectorants, or antihistamines or a mixture or combination thereof. Suitable non-limiting nasal decongestants comprise pseudoephedrine, phenylephrine, and phenylpropanolamine or a mixture or combination thereof. Suitable non-limiting antihistamines comprise astemizole, azelastine, azatadine, brompheniramine, carbinoxamine, cetirizine, chlorpheniramine, clemastine, cyproheptadine, desloratadine, dexbrompheniramine, dexchlorpheniramine, diphenhydramine, fexofenadine, hydroxyzine, levocetirizine, loratadine, phenindamine, pheniramine, phenyltoloxamine, promethazine, pyrilamine, terfenadine, tripelennamine, triprolidine, or a mixture or combination thereof. Suitable non-limiting antitussives comprise acetyl dihydrocodeine, benproperine, benzonatate, benzylmorphine, bibenzonium bromide, butamirate, butorphanol, carbetapentane, chlophedianol, clobutinol, clofedanol, cloperastine, codeine, dextromethorphan, diacetylmorphine, dibunate, dihydrocodeine, dimemorfan, dimethoxanate, diphenhydramine, dropropizine, droxypropine, ethylmorphine, fedrilate, glaucine, hydrocodone, hydromorphone, isoaminile, laudanum, levodropropizine, levomethadone, levopropoxyphene, meprotixol, methadone, morclofone, nepinalone, nicocodine, nicodicodine, normethadone, noscapine, oxeladin, oxolamine, pentoxyverine, pholcodine, pipazetate, piperidione, prenoxdiazine, tipepidine, zipeprol, or a mixture or combination thereof. Suitable non-limiting expectorants and mucolytics comprise acetylcysteine, althea root, ambroxol, antimony pentasulfide, bromhexine, carbocisteine, cineole, combinations, combinations, creosote, dembrexine hydrochloride, domiodol, dornase alfa, eprazinone, erdosteine, guaiacolsulfonate, guaifenesin, hedera helicis folium, ipecacuanha, letosteine, levo verbenone, mannitol, mesna, neltenexine, potassium iodide, senega, sobrerol, stepronin, tiopronin, tyloxapol, or a mixture or combination thereof.

Another embodiment described herein is a method for treating, ameliorating the symptoms of, or delaying the onset of a medical condition by providing a subject in need thereof with a pharmaceutical composition comprising a soft capsule, as described herein, comprising a pharmaceutical ingredient or ingredients. As used herein, a medical condition can comprise any actual or suspected disease, disorder, or condition that a subject may seek medical care therefor. One embodiment described herein is method of treating, ameliorating the symptoms of, or delaying the onset of a medical condition of includes administering a pharmaceutical ingredient having a desired therapeutic or biological activity or suspected of having a desired therapeutic or biological activity in a subject in need thereof.

In another embodiment, the pharmaceutical compositions described herein comprise pharmaceutically acceptable salts of any of the above mentioned active pharmaceutical ingredients. The term “pharmaceutically acceptable salts” of an active pharmaceutical ingredient includes alkali metal salts such as, for example, sodium or potassium salts, alkaline earth metal salts such as, for example, calcium and magnesium salts, and salts with organic or inorganic acid such as, for example, hydrochloric acid, hydrobromic acid, nitric acid, sulfuric acid, phosphoric acid, citric acid, formic acid, maleic acid, succinic acid, tartaric acid, methanesulphonic acid, toluenesulphonic acid etc. In another embodiment, the active pharmaceutical ingredient may also be in the form of pharmaceutically acceptable salts, uncharged or charged molecules, molecular complexes, solvates, or anhydrates thereof, and, if relevant, single isomers, enantiomers, racemic mixtures, or mixtures thereof.

In another embodiment, the active pharmaceutical ingredient may be in any of its crystalline, polymorphous, semi-crystalline, amorphous or polyamorphous forms or mixtures thereof.

In one embodiment, the pharmaceutical composition described herein, comprises an active pharmaceutical ingredient of about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 550 mg, about 600 mg, about 650 mg, about 700 mg, about 750 mg, or about 800 mg.

In one embodiment, the pharmaceutical composition described herein, comprises an active pharmaceutical ingredient in the range of about 50 mg to about 200 mg, about 75 mg to about 200 mg, about 100 mg to about 200 mg, about 125 mg to about 200 mg, about 150 mg to about 200 mg, or about 175 mg to about 200 mg, including all iterations of integers within the specified ranges above.

In another embodiment, the pharmaceutical composition described herein, comprises an active pharmaceutical ingredient in the range of about 50 mg to about 400 mg, about 100 mg to about 400 mg, about 150 mg to about 400 mg, about 200 mg to about 400 mg, about 250 mg to about 400 mg, about 300 mg to about 400 mg, or about 350 mg to about 400 mg, including all iterations of integers within the specified ranges above.

In another embodiment, the pharmaceutical composition described herein, comprises an active pharmaceutical ingredient in the range of about 50 mg to about 600 mg, about 100 mg to about 600 mg, about 150 mg to about 600 mg, about 200 mg to about 600 mg, about 250 mg to about 600 mg, about 300 mg to about 600 mg, about 350 mg to about 600 mg, about 400 mg to about 600 mg, about 450 mg to about 600 mg, about 500 mg to about 600 mg, or about 550 mg to about 600 mg, including all iterations of integers within the specified ranges above.

In another embodiment, the pharmaceutical composition described herein, comprises an active pharmaceutical ingredient in the range of about 50 mg to about 800 mg, 100 mg to about 800 mg about 150 mg to about 800 mg, about 200 mg to about 800 mg, about 250 mg to about 800 mg, about 300 mg to about 800 mg, about 350 mg to about 800 mg, about 400 mg to about 800 mg, about 400 mg to about 800 mg, about 450 mg to about 800 mg, about 500 mg to about 800 mg about 550 mg to about 800 mg, about 600 mg to about 800 mg, about 650 mg to about 800 mg, about 700 mg to about 800 mg, or about 750 mg to about 800 mg, including all iterations of integers within the specified ranges above.

The dosage form can be administered, for example, 1×, 2×, 3×, 4×, 5×, 6×, 7×, or 8×, per day. One or more dosage form can be administered, for example, for 1, 2, 3, 4, 5, 6, 7 days, or even longer. One or more dosage forms can be administered, for example, for 1, 2, 3, 4 weeks, or even longer. One or more dosage forms can be administered, for example, for 1, 2, 3, 4, 5, 6, 7, 8, 9, 10, 11, 12 months, or even longer. One or more dosage forms can be administered until the patient, subject, mammal, mammal in need thereof, human, or human in need thereof, does not require treatment, prophylaxis, or amelioration of any disease or condition such as, for example, pain. In some aspects, the dosage form may be co-administered with other pharmaceutical compositions until the patient, subject, mammal, mammal in need thereof, human, or human in need thereof, does not require treatment, prophylaxis, or amelioration of any disease or condition.

In one embodiment described herein, the soft capsules described herein comprise an active pharmaceutical ingredient comprising ibuprofen or a pharmaceutically acceptable salt form thereof, including but not limited to ibuprofen sodium or other ibuprofen salts, and salt hydrates. In one embodiment, ibuprofen is present in its salt form. In another embodiment, ibuprofen is present as ibuprofen sodium dihydrate. As used herein, “ibuprofen” refers all possible salt forms of the active pharmaceutical ingredient if a particular salt is not specified.

Without being bound by any theory, it is thought that ionized ibuprofen, e.g., ibuprofen salts such as ibuprofen sodium dihydrate, is absorbed more rapidly in the stomach because it is soluble in aqueous and acidic environments, such as the stomach, when compared to ibuprofen free acid. See Sõrgel et al., Int. J. Clin. Pharmacol. Then 43(3): 140-149 (2005), which is incorporated by reference herein for such teachings. Because ibuprofen free acid and salt forms are both substantially absorbed, the total exposure to ibuprofen is the same; thus both forms are equally safe and demonstrate virtually identical clearance rates.

In one embodiment described herein, the dose of ibuprofen is about 50 mg to about 800 mg, including all integers within the specified range. In one aspect, the dose of ibuprofen is about 50 mg. In another aspect, the dose of ibuprofen is about 75 mg. In another aspect, the dose of ibuprofen is about 100 mg. In another aspect, the dose of ibuprofen is about 125 mg. In another aspect, the dose of ibuprofen is about 150 mg. In another aspect, the dose of ibuprofen is about 175 mg. In another aspect, the dose of ibuprofen is about 200 mg. In another aspect, the dose of ibuprofen is about 225 mg. In another aspect, the dose of ibuprofen is about 250 mg. In another aspect, the dose of ibuprofen is about 300 mg. In another aspect, the dose of ibuprofen is about 350 mg. In another aspect, the dose of ibuprofen is about 400 mg. In another aspect, the dose of ibuprofen is about 450 mg. In another aspect, the dose of ibuprofen is about 500 mg. In another aspect, the dose of ibuprofen is about 600 mg. In another aspect, the dose of ibuprofen is about 650 mg. In another aspect, the dose of ibuprofen is about 700 mg. In another aspect, the dose of ibuprofen is about 650 mg. In another aspect, the dose of ibuprofen is about 800 mg.

In one aspect, the total dosage of ibuprofen administered in a 24-hour period is about 100 mg. In another aspect, the total dosage of ibuprofen administered in a 24-hour period is about 200 mg. In another aspect, the total dosage of ibuprofen administered in a 24-hour period is about 400 mg. In another aspect, the total dosage of ibuprofen administered in a 24-hour period is about 600 mg. In another aspect, the total dosage of ibuprofen administered in a 24-hour period is about 800 mg. In another aspect, the total dosage of ibuprofen administered in a 24-hour period is about 1000 mg. In another aspect, the total dosage of ibuprofen administered in a 24-hour period is about 1200 mg. In another aspect, the total dosage of ibuprofen administered in a 24-hour period is about 1400 mg. In another aspect, the total dosage of ibuprofen administered in a 24-hour period is about 1600 mg. In another aspect, the total dosage of ibuprofen administered in a 24-hour period is about 1800 mg. In another aspect, the total dosage of ibuprofen administered in a 24-hour period is about 2000 mg. In another aspect, the total dosage of ibuprofen administered in a 24-hour period is about 2200 mg. In another aspect, the total dosage of ibuprofen administered in a 24-hour period is about 2400 mg. In another aspect, the total dosage of ibuprofen administered in a 24-hour period is about 2600 mg. In another aspect, the total dosage of ibuprofen administered in a 24-hour period is about 2800 mg. In another aspect, the total dosage of ibuprofen administered in a 24-hour period is about 3000 mg. In another aspect, the total dosage of ibuprofen administered in a 24-hour period is about 3200 mg.

In one embodiment, the total dosage of ibuprofen administered in a 24-hour period is about 100 mg to about 3200 mg per 24-hour period including all iterations of integers within the specified range. In another embodiment, the total dosage of ibuprofen administered in a 24-hour period is about 200 mg to about 400 mg per 24-hour period including all iterations of integers within the specified range. In another embodiment, the total dosage of ibuprofen administered in a 24-hour period is about 400 mg to about 800 mg per 24-hour period including all iterations of integers within the specified range. In another embodiment, the total dosage of ibuprofen administered in a 24-hour period is about 800 mg to about 1600 mg per 24-hour period including all iterations of integers within the specified range. In another embodiment, the total dosage of ibuprofen administered in a 24-hour period is about 1600 mg to about 2400 mg per 24-hour period including all iterations of integers within the specified range. In another embodiment, the total dosage of ibuprofen administered in a 24-hour period is about 2400 mg to about 3200 mg per 24-hour period including all iterations of integers within the specified range.

In another embodiment, the total dosage of ibuprofen administered in a 24-hour period is about 200 mg to about 3200 mg and is effective for the treatment of pain is administered in equal daily doses. In one aspect, 200 mg of ibuprofen is administered 2 times daily or 400 mg 1 time daily for a total of 400 mg to reach a desired therapeutic efficacy. In another aspect, 200 mg of ibuprofen is administered 1 times daily or 400 mg 2 times daily, or 800 mg 1 times daily for a total of 800 mg to reach a desired therapeutic efficacy. In another aspect, 200 mg of ibuprofen is administered 6 times daily or 400 mg 3 times daily for a total of 1200 mg to reach a desired therapeutic efficacy. In another aspect, 400 mg of ibuprofen is administered 6 times daily or 800 mg 3 times daily for a total of 2400 mg to reach a desired therapeutic efficacy. In another aspect, 400 mg of ibuprofen is administered 8 times daily or 800 mg 4 times daily for a total of 3200 mg to reach a desired therapeutic efficacy.

In one embodiment, the dosage can contain an amount of ibuprofen effective for treatment, amelioration, prophylaxis, or reducing the onset of or symptoms of mild, moderate, or severe pain.

In one embodiment, the dosage can contain an amount of ibuprofen effective for treatment, amelioration, prophylaxis, or reducing the onset of or symptoms of mild, moderate, or severe inflammation.

In another embodiment, the dosage can contain an amount of ibuprofen effective for treatment, amelioration, prophylaxis, or reducing the onset of or symptoms of mild, moderate, or severe fever.

In one embodiment, the dosage can contain an amount of ibuprofen effective for treatment, amelioration, prophylaxis, or reducing the onset of or symptoms of mild, moderate, or severe pain and inflammation.

In one embodiment, the dosage can contain an amount of ibuprofen effective for treatment, amelioration, prophylaxis, or reducing the onset of or symptoms of mild, moderate, or severe pain or inflammation stemming from bacterial or viral infections, including, but not limited to common colds and influenza.

In another embodiment, the dosage can contain an amount of ibuprofen effective for treatment, amelioration, prophylaxis, or reducing the onset of or symptoms of mild, moderate, or severe pain or inflammation stemming from tendonitis.

In another embodiment, the dosage can contain an amount of ibuprofen effective for treatment, amelioration, prophylaxis, or reducing the onset of or symptoms of mild, moderate, or severe pain or inflammation stemming from bursitis.

In another embodiment, the dosage can contain an amount of ibuprofen effective for treatment, amelioration, prophylaxis, or reducing the onset of or symptoms of mild, moderate, or severe pain or inflammation stemming from chronic neuropathies.

In another embodiment, the dosage can contain an amount of ibuprofen effective for treatment, amelioration, prophylaxis, or reducing the onset of or symptoms of mild, moderate, or severe pain or inflammation stemming from shingles.

In another embodiment, the dosage can contain an amount of ibuprofen effective for treatment, amelioration, prophylaxis, or reducing the onset of or symptoms of mild, moderate, or severe pain or inflammation stemming from chronic sports or traumatic injuries.

In another embodiment, the dosage can contain an amount of ibuprofen effective for treatment, amelioration, prophylaxis, or reducing the onset of or symptoms of mild, moderate, or severe pain or inflammation stemming from chronic malignancies and/or cancer.

In another embodiment, the dosage can contain an amount of ibuprofen effective for treatment, amelioration, prophylaxis, or reducing the onset of or symptoms of mild, moderate, or severe pain or inflammation stemming from chronic radiculopathy.

In another embodiment, the dosage can contain an amount of ibuprofen effective for treatment, amelioration, prophylaxis, or reducing the onset of or symptoms of mild, moderate, or severe pain or inflammation stemming from chronic sciatica.

In another embodiment, the dosage can contain an amount of ibuprofen effective for treatment, amelioration, prophylaxis, or reducing the onset of or symptoms of pain or inflammation associated with kidney stones.

In another embodiment, the dosage can contain an amount of ibuprofen effective for treatment, amelioration, prophylaxis, or reducing the onset of or symptoms of menstrual pain or inflammation.

In another embodiment, the dosage can contain an amount of ibuprofen effective for treatment, amelioration, prophylaxis, or reducing the onset of or symptoms of dysmenorrhea.

In another embodiment, the dosage can contain an amount of ibuprofen effective for treatment, amelioration, prophylaxis, or reducing the onset of or symptoms of pain or inflammation associated with endometriosis.

In another embodiment, the dosage can contain an amount of ibuprofen effective for treatment, amelioration, prophylaxis, or reducing the onset of or symptoms of acute migraines.

In another embodiment, the dosage can contain an amount of ibuprofen effective for treatment, amelioration, prophylaxis, or reducing the onset of or symptoms of osteoarthritis.

In another embodiment, the dosage can contain an amount of ibuprofen effective for treatment, amelioration, prophylaxis, or reducing the onset of or symptoms of rheumatoid arthritis.

In another embodiment, the dosage can contain an amount of ibuprofen effective for treatment, amelioration, prophylaxis, or reducing the onset of or symptoms of ankylosing spondylitis.

In another embodiment, the dosage can contain an amount of ibuprofen effective for treatment, amelioration, prophylaxis, or reducing the onset of or symptoms of spondylarthritis.

In another embodiment, the dosage can contain an amount of ibuprofen effective for treatment, amelioration, prophylaxis, or reducing the onset of or symptoms of gout.

In another embodiment, the dosage can contain an amount of ibuprofen and an amount of one or more nasal decongestants, antitussives, expectorants, or antihistamines or a mixture or combination thereof for the treatment, amelioration, prophylaxis, or reducing the onset of or symptoms of a cough or cold.

The concentration of the active pharmaceutical ingredient in the pharmaceutical composition depends on the specific active pharmaceutical ingredient, the disease to be treated, the condition of the patient, the age, and gender of the patient, etc. The active pharmaceutical ingredient may be a well-known active pharmaceutical ingredient and a person having ordinary skill in the art will be able to find information as to the dosage of each active drug substance and, accordingly, will know how to determine the amount of each active drug substance in the pharmaceutical composition.

Another embodiment described herein is a method for treating, retarding the progression of, prophylaxis of, delaying the onset of, ameliorating, or reducing the symptoms of pain, inflammation, or fever comprising the administration of a therapeutically effective amount of ibuprofen sodium comprising any one of the pharmaceutical compositions described herein to a subject with pain, wherein the administration is sufficient to achieve a reduction of pain, reduction of inflammation, or reduction of fever relative to baseline in the subject without substantially inducing one or more of abdominal pain, acid or sour stomach, belching, bloating, cloudy urine, decrease in amount of urine, decrease in urine output or decrease in urine-concentrating ability, diarrhea, difficulty having a bowel movement (stool), excess air or gas in stomach or intestines, full feeling, heartburn, indigestion, itching skin, nausea, noisy, rattling breathing, pain or discomfort in chest, upper stomach, or throat, pale skin, passing gas, rash with flat lesions or small raised lesions on the skin, shortness of breath, swelling of face, fingers, hands, feet, lower legs, or ankles, troubled breathing at rest, troubled breathing with exertion, unusual bleeding or bruising, unusual tiredness or weakness, vomiting, or weight gain.

In one aspect, after administration of any one the pharmaceutical compositions described herein, the subject experiences the side effects described herein at a rate of less than about 10%. In another aspect, the subject experiences the side effects described herein at a rate of less than about 2%, about 5%, about 10%, about 15%, about 20%, about 25%, about 30%, about 35%, about 45%, about 50%, about 60%, about 70%, about 80%, or about 90%.

In one embodiment described herein, a subject is administered a pharmaceutical composition comprising a dose of ibuprofen of about 200 mg to about 800 mg. In one aspect, the pharmaceutical composition comprises an immediate release form of ibuprofen.

In one embodiment described herein, the pharmaceutical composition comprises a dose of ibuprofen of about 200 mg, wherein subjects administered an initial dosage of said composition exhibit a mean plasma ibuprofen Tₘₐₓ ranging from 0.7 h to about 1.75 h, including all iterations of integers within the specified range. In another embodiment, the pharmaceutical composition comprises a dose of ibuprofen of about 200 mg, wherein subjects administered an initial dosage of said composition exhibit a mean plasma ibuprofen Cₘₐₓ ranging from about 15 mg/L to about 29 mg/L, including all iterations of integers within the specified range. In another embodiment, the pharmaceutical composition comprises a dose of ibuprofen of about 200 mg, wherein subjects administered an initial dosage of said composition exhibit a mean plasma ibuprofen AUC_{0→12} ranging from about 62 h·mg/L to about 77 h·mg/L, including all iterations of integers within the specified range. In another embodiment, the pharmaceutical composition comprises a dose of ibuprofen of about 200 mg, wherein subjects administered an initial dosage of said composition exhibit a mean plasma ibuprofen AUC_{0→∞} ranging from about 66 h·mg/L to about 79 h·mg/L, including all iterations of integers within the specified range. In another embodiment, the pharmaceutical composition comprises a dose of ibuprofen of about 200 mg, wherein subjects administered an initial dosage of said composition exhibit a mean plasma ibuprofen half-life (t½) ranging from about 2.3 h to about 2.4 h, including all iterations of integers within the specified range. In another embodiment, the pharmaceutical composition comprises a dose of ibuprofen of about 200 mg, wherein subjects administered an initial dosage of said composition exhibit a mean plasma ibuprofen terminal elimination rate constant (λ_{z}) ranging from about 0.29 h⁻¹ to about 0.31 h⁻¹, including all iterations of integers within the specified range.

In another embodiment described herein, the pharmaceutical composition comprises a dose of ibuprofen of about 200 mg, wherein subjects under fasting conditions administered an initial dosage of said composition exhibit a mean plasma ibuprofen Tₘₐₓ of about 0.7 h. In another embodiment, the pharmaceutical composition comprises a dose of ibuprofen of about 200 mg, wherein subjects under fasting conditions administered an initial dosage of said composition exhibit a mean plasma ibuprofen Cₘₐₓ of about 28 mg/L. In another embodiment, the pharmaceutical composition comprises a dose of ibuprofen of about 200 mg, wherein subjects under fasting conditions administered an initial dosage of said composition exhibit a mean plasma ibuprofen AUC_{0→12} of about 77 h·mg/L. In another embodiment, the pharmaceutical composition comprises a dose of ibuprofen of about 200 mg, wherein subjects under fasting conditions administered an initial dosage of said composition exhibit a mean plasma ibuprofen AUC_{0→∞} of about 79 h·mg/L. In another embodiment, the pharmaceutical composition comprises a dose of ibuprofen of about 200 mg, wherein subjects under fasting conditions administered an initial dosage of said composition exhibit a mean plasma ibuprofen half-life (t½) of about 2.4 h. In another embodiment, the pharmaceutical composition comprises a dose of ibuprofen of about 200 mg, wherein subjects under fasting conditions administered an initial dosage of said composition exhibit a mean plasma ibuprofen terminal elimination rate constant (λ_{z}) of about 0.29 h⁻¹.

In another embodiment described herein, the pharmaceutical composition comprises a dose of ibuprofen of about 200 mg, wherein subjects under fed conditions administered an initial dosage of said composition exhibit a mean plasma ibuprofen Tₘₐₓ of about 1.8 h. In another embodiment, the pharmaceutical composition comprises a dose of ibuprofen of about 200 mg, wherein subjects under fed conditions administered an initial dosage of said composition exhibit a mean plasma ibuprofen Cₘₐₓ of about 15 mg/L. In another embodiment, the pharmaceutical composition comprises a dose of ibuprofen of about 200 mg, wherein subjects under fed conditions administered an initial dosage of said composition exhibit a mean plasma ibuprofen AUC_{0→12} of about 62 h·mg/L. In another embodiment, the pharmaceutical composition comprises a dose of ibuprofen of about 200 mg, wherein subjects under fed conditions administered an initial dosage of said composition exhibit a mean plasma ibuprofen AUC_{0→∞} of about 66 h·mg/L. In another embodiment, the pharmaceutical composition comprises a dose of ibuprofen of about 200 mg, wherein subjects under fed conditions administered an initial dosage of said composition exhibit a mean plasma ibuprofen half-life (t½) of about 2.3 h. In another embodiment, the pharmaceutical composition comprises a dose of ibuprofen of about 200 mg, wherein subjects under fed conditions administered an initial dosage of said composition exhibit a mean plasma ibuprofen terminal elimination rate constant (λ_{z}) of about 0.31 h⁻¹.

In another embodiment, the pharmaceutical composition comprises a dose of ibuprofen of about 200 mg to about 800 mg, wherein subjects administered an initial dosage exhibit a mean plasma ibuprofen Cₘₐₓ ranging from about 8 mg/L to about 80 mg/L, including all iterations of integers within the specified range. In another aspect, the pharmaceutical composition comprises a dose of ibuprofen of about 200 mg to about 800 mg, wherein subjects administered an initial dosage exhibit a mean plasma ibuprofen Cₘₐₓ ranging from about 8 mg/L to about 20 mg/L, including all iterations of integers within the specified range. In another aspect, the pharmaceutical composition comprises a dose of ibuprofen of about 200 mg to about 800 mg, wherein subjects administered an initial dosage exhibit a mean plasma ibuprofen Cₘₐₓ ranging from about 20 mg/L to about 30 mg/L, including all iterations of integers within the specified range. In another aspect, the pharmaceutical composition comprises a dose of ibuprofen of about 200 mg to about 800 mg, wherein subjects administered an initial dosage exhibit a mean plasma ibuprofen Cₘₐₓ ranging from about 30 mg/L to about 40 mg/L, including all iterations of integers within the specified range. In another aspect, the pharmaceutical composition comprises a dose of ibuprofen of about 200 mg to about 800 mg, wherein subjects administered an initial dosage exhibit a mean plasma ibuprofen Cₘₐₓ ranging from about 40 mg/L to about 50 mg/L, including all iterations of integers within the specified range. In another aspect, the pharmaceutical composition comprises a dose of ibuprofen of about 200 mg to about 800 mg, wherein subjects administered an initial dosage exhibit a mean plasma ibuprofen Cₘₐₓ ranging from about 50 mg/L to about 60 mg/L, including all iterations of integers within the specified range. In another aspect, the pharmaceutical composition comprises a dose of ibuprofen of about 200 mg to about 800 mg, wherein subjects administered an initial dosage exhibit a mean plasma ibuprofen Cₘₐₓ ranging from about 60 mg/L to about 70 mg/L, including all iterations of integers within the specified range. In another aspect, the pharmaceutical composition comprises a dose of ibuprofen of about 200 mg to about 800 mg, wherein subjects administered an initial dosage exhibit a mean plasma ibuprofen Cₘₐₓ ranging from about 70 mg/L to about 80 mg/L, including all iterations of integers within the specified range. In another aspect, the pharmaceutical composition comprises a dose of ibuprofen of about 200 mg to about 800 mg, wherein subjects administered an initial dosage exhibit a mean plasma ibuprofen Cₘₐₓ of at least 10 mg/L, at least 15 mg/L, at least 20 mg/L, at least 25 mg/L, at least 30 mg/L, at least 35 mg/L, at least 40 mg/L, at least 45 mg/L, at least 50 mg/L, at least 55 mg/L, at least 60 mg/L, at least 65 mg/L, at least 70 mg/L, at least 75 mg/L, or at least 80 mg/L.

In another embodiment, the pharmaceutical composition comprises a dose of ibuprofen of about 200 mg to about 800 mg, wherein subjects administered an initial dosage exhibit a plasma ibuprofen AUC L ranging from about 50 h·mg/L to about 500 h·mg/L, including all iterations of integers within the specified range. In another embodiment, the pharmaceutical composition comprises a dose of ibuprofen of about 200 mg to about 800 mg, wherein subjects administered an initial dosage exhibit a plasma ibuprofen AUC L ranging from about 50 h·mg/L to about 100 h·mg/L, including all iterations of integers within the specified range. In another embodiment, the pharmaceutical composition comprises a dose of ibuprofen of about 200 mg to about 800 mg, wherein subjects administered an initial dosage exhibit a plasma ibuprofen AUC L ranging from about 100 h·mg/L to about 150 h·mg/L, including all iterations of integers within the specified range. In another embodiment, the pharmaceutical composition comprises a dose of ibuprofen of about 200 mg to about 800 mg, wherein subjects administered an initial dosage exhibit a plasma ibuprofen AUC L ranging from about 150 h·mg/L to about 200 h·mg/L, including all iterations of integers within the specified range. In another embodiment, the pharmaceutical composition comprises a dose of ibuprofen of about 200 mg to about 800 mg, wherein subjects administered an initial dosage exhibit a plasma ibuprofen AUC L ranging from about 200 h·mg/L to about 250 h·mg/L, including all iterations of integers within the specified range. In another embodiment, the pharmaceutical composition comprises a dose of ibuprofen of about 200 mg to about 800 mg, wherein subjects administered an initial dosage exhibit a plasma ibuprofen AUC L ranging from about 250 h·mg/L to about 300 h·mg/L, including all iterations of integers within the specified range. In another embodiment, the pharmaceutical composition comprises a dose of ibuprofen of about 200 mg to about 800 mg, wherein subjects administered an initial dosage exhibit a plasma ibuprofen AUC_{L} ranging from about 300 h·mg/L to about 350 h·mg/L, including all iterations of integers within the specified range. In another embodiment, the pharmaceutical composition comprises a dose of ibuprofen of about 200 mg to about 800 mg, wherein subjects administered an initial dosage exhibit a plasma ibuprofen AUC L ranging from about 350 h·mg/L to about 500 h·mg/L, including all iterations of integers within the specified range. In another embodiment, the pharmaceutical composition comprises a dose of ibuprofen of about 200 mg to about 800 mg, wherein subjects administered an initial dosage exhibit a plasma ibuprofen AUC L of at least about 50 h·mg/L, at least about 75 h·mg/L, at least about 100 h·mg/L, at least about 125 h·mg/L, at least about 150 h·mg/L, at least about 175 h·mg/L, at least about 200 h·mg/L, at least about 225 h·mg/L, at least about 250 h·mg/L, at least about 275 h·mg/L, at least about 300 h·mg/L, at least about 325 h·mg/L, at least about 350 h·mg/L, at least about 375 h·mg/L, at least about 400 h·mg/L, at least about 425 h·mg/L, at least about 450 h·mg/L, at least about 475 h·mg/L, or at least about 500 h·mg/L.

In another embodiment, the pharmaceutical composition comprises a dose of ibuprofen of about 200 mg to about 800 mg, wherein subjects administered an initial dosage exhibit a minimum plasma ibuprofen Tₘₐₓ under fasting conditions ranging from about 5 minutes to about 60 minutes, including all iterations of integers within the specified range. In another embodiment, the pharmaceutical composition comprises a dose of ibuprofen of about 200 mg to about 800 mg, wherein subjects administered an initial dosage exhibit a minimum plasma ibuprofen Tₘₐₓ ranging from about 10 minutes to about 20 minutes, including all iterations of integers within the specified range. In another embodiment, the pharmaceutical composition comprises a dose of ibuprofen of about 200 mg to about 800 mg, wherein subjects administered an initial dosage exhibit a minimum plasma ibuprofen Tₘₐₓ ranging from about 20 minutes to about 30 minutes, including all iterations of integers within the specified range. In another embodiment, the pharmaceutical composition comprises a dose of ibuprofen of about 200 mg to about 800 mg, wherein subjects administered an initial dosage exhibit a minimum plasma ibuprofen Tₘₐₓ ranging from about 30 minutes to about 40 minutes, including all iterations of integers within the specified range. In another embodiment, the pharmaceutical composition comprises a dose of ibuprofen of about 200 mg to about 800 mg, wherein subjects administered an initial dosage exhibit a minimum plasma ibuprofen Tₘₐₓ ranging from about 40 minutes to about 50 minutes, including all iterations of integers within the specified range. In another embodiment, the pharmaceutical composition comprises a dose of ibuprofen of about 200 mg to about 800 mg, wherein subjects administered an initial dosage exhibit a minimum plasma ibuprofen Tₘₐₓ ranging from about 50 minutes to about 60 minutes, including all iterations of integers within the specified range. In another embodiment, the pharmaceutical composition comprises a dose of ibuprofen of about 200 mg to about 800 mg, wherein subjects administered an initial dosage exhibit a minimum plasma ibuprofen Tₘₐₓ of about 5 min, about 10 min, about 15 min, about 20 min, about 25 min, about 30 min, about 35 min, about 40 min, about 45 min, about 50 min, about 55 min, or about 60 min.

In another embodiment, the pharmaceutical composition comprises a dose of ibuprofen of about 200 mg to about 800 mg, wherein subjects administered an initial dosage exhibit a minimum plasma ibuprofen Tₘₐₓ under fed conditions ranging from about 10 minutes to about 60 minutes, including all iterations of integers within the specified range. In another embodiment, the pharmaceutical composition comprises a dose of ibuprofen of about 200 mg to about 800 mg, wherein subjects administered an initial dosage exhibit a minimum plasma ibuprofen Tₘₐₓ under fed conditions ranging from about 10 minutes to about 20 minutes, including all iterations of integers within the specified range. In another embodiment, the pharmaceutical composition comprises a dose of ibuprofen of about 200 mg to about 800 mg, wherein subjects administered an initial dosage exhibit a minimum plasma ibuprofen Tₘₐₓ under fed conditions ranging from about 20 minutes to about 30 minutes, including all iterations of integers within the specified range. In another embodiment, the pharmaceutical composition comprises a dose of ibuprofen of about 200 mg to about 800 mg, wherein subjects administered an initial dosage exhibit a minimum plasma ibuprofen Tₘₐₓ under fed conditions ranging from about 30 minutes to about 40 minutes, including all iterations of integers within the specified range. In another embodiment, the pharmaceutical composition comprises a dose of ibuprofen of about 200 mg to about 800 mg, wherein subjects administered an initial dosage exhibit a minimum plasma ibuprofen Tₘₐₓ under fed conditions ranging from about 40 minutes to about 50 minutes, including all iterations of integers within the specified range. In another embodiment, the pharmaceutical composition comprises a dose of ibuprofen of about 200 mg to about 800 mg, wherein subjects administered an initial dosage exhibit a minimum plasma ibuprofen Tₘₐₓ under fed conditions ranging from about 50 minutes to about 60 minutes, including all iterations of integers within the specified range. In another embodiment, the pharmaceutical composition comprises a dose of ibuprofen of about 200 mg to about 800 mg, wherein subjects administered an initial dosage under fed conditions exhibit a minimum plasma ibuprofen Tₘₐₓ of about 5 min, about 10 min, about 15 min, about 20 min, about 25 min, about 30 min, about 35 min, about 40 min, about 45 min, about 50 min, about 55 min, or about 60 min.

In another embodiment, the pharmaceutical composition comprises a dose of ibuprofen of about 200 mg to about 800 mg, wherein subjects administered an initial dosage exhibit a mean plasma ibuprofen Tₘₐₓ under fasting conditions ranging from about 5 minutes to about 60 minutes, including all iterations of integers within the specified range. In another embodiment, the pharmaceutical composition comprises a dose of ibuprofen of about 200 mg to about 800 mg, wherein subjects administered an initial dosage exhibit a mean plasma ibuprofen Tₘₐₓ ranging from about 10 minutes to about 20 minutes, including all iterations of integers within the specified range. In another embodiment, the pharmaceutical composition comprises a dose of ibuprofen of about 200 mg to about 800 mg, wherein subjects administered an initial dosage exhibit a mean plasma ibuprofen Tₘₐₓ ranging from about 20 minutes to about 30 minutes, including all iterations of integers within the specified range. In another embodiment, the pharmaceutical composition comprises a dose of ibuprofen of about 200 mg to about 800 mg, wherein subjects administered an initial dosage exhibit a mean plasma ibuprofen Tₘₐₓ ranging from about 30 minutes to about 40 minutes, including all iterations of integers within the specified range. In another embodiment, the pharmaceutical composition comprises a dose of ibuprofen of about 200 mg to about 800 mg, wherein subjects administered an initial dosage exhibit a mean plasma ibuprofen Tₘₐₓ ranging from about 40 minutes to about 50 minutes, including all iterations of integers within the specified range. In another embodiment, the pharmaceutical composition comprises a dose of ibuprofen of about 200 mg to about 800 mg, wherein subjects administered an initial dosage exhibit a mean plasma ibuprofen Tₘₐₓ ranging from about 50 minutes to about 60 minutes, including all iterations of integers within the specified range. In another embodiment, the pharmaceutical composition comprises a dose of ibuprofen of about 200 mg to about 800 mg, wherein subjects administered an initial dosage exhibit a mean plasma ibuprofen Tₘₐₓ of about 5 min, about 10 min, about 15 min, about 20 min, about 25 min, about 30 min, about 35 min, about 40 min, about 45 min, about 50 min, about 55 min, or about 60 min.

In another embodiment, the pharmaceutical composition comprises a dose of ibuprofen of about 200 mg to about 800 mg, wherein subjects administered an initial dosage exhibit a mean plasma ibuprofen Tₘₐₓ under fed conditions ranging from about 30 minutes to about 180 minutes, including all iterations of integers within the specified range. In another embodiment, the pharmaceutical composition comprises a dose of ibuprofen of about 200 mg to about 800 mg, wherein subjects administered an initial dosage exhibit a mean plasma ibuprofen Tₘₐₓ under fed conditions ranging from about 30 minutes to about 50 minutes, including all iterations of integers within the specified range. In another embodiment, the pharmaceutical composition comprises a dose of ibuprofen of about 200 mg to about 800 mg, wherein subjects administered an initial dosage exhibit a mean plasma ibuprofen Tₘₐₓ under fed conditions ranging from about 50 minutes to about 70 minutes, including all iterations of integers within the specified range. In another embodiment, the pharmaceutical composition comprises a dose of ibuprofen of about 200 mg to about 800 mg, wherein subjects administered an initial dosage exhibit a mean plasma ibuprofen Tₘₐₓ under fed conditions ranging from about 70 minutes to about 90 minutes, including all iterations of integers within the specified range. In another embodiment, the pharmaceutical composition comprises a dose of ibuprofen of about 200 mg to about 800 mg, wherein subjects administered an initial dosage exhibit a mean plasma ibuprofen Tₘₐₓ under fed conditions ranging from about 90 minutes to about 120 minutes, including all iterations of integers within the specified range. In another embodiment, the pharmaceutical composition comprises a dose of ibuprofen of about 200 mg to about 800 mg, wherein subjects administered an initial dosage exhibit a mean plasma ibuprofen Tₘₐₓ under fed conditions ranging from about 120 minutes to about 150 minutes, including all iterations of integers within the specified range. In another embodiment, the pharmaceutical composition comprises a dose of ibuprofen of about 200 mg to about 800 mg, wherein subjects administered an initial dosage exhibit a mean plasma ibuprofen Tₘₐₓ under fed conditions ranging from about 120 minutes to about 150 minutes, including all iterations of integers within the specified range. In another embodiment, the pharmaceutical composition comprises a dose of ibuprofen of about 200 mg to about 800 mg, wherein subjects administered an initial dosage exhibit a mean plasma ibuprofen Tₘₐₓ under fed conditions ranging from about 150 minutes to about 180 minutes, including all iterations of integers within the specified range. In another embodiment, the pharmaceutical composition comprises a dose of ibuprofen of about 200 mg to about 800 mg, wherein subjects administered an initial dosage exhibit a mean plasma ibuprofen Tₘₐₓ under fed conditions ranging from about 180 minutes to about 210 minutes, including all iterations of integers within the specified range. In another embodiment, the pharmaceutical composition comprises a dose of ibuprofen of about 200 mg to about 800 mg, wherein subjects administered an initial dosage exhibit a mean plasma ibuprofen Tₘₐₓ under fed conditions ranging from about 210 minutes to about 250 minutes, including all iterations of integers within the specified range. In another embodiment, the pharmaceutical composition comprises a dose of ibuprofen of about 200 mg to about 800 mg, wherein subjects administered an initial dosage exhibit a mean plasma ibuprofen Tₘₐₓ under fed conditions ranging from about 250 minutes to about 300 minutes, including all iterations of integers within the specified range. In another embodiment, the pharmaceutical composition comprises a dose of ibuprofen of about 200 mg to about 800 mg, wherein subjects administered an initial dosage under fed conditions exhibit a mean plasma ibuprofen Tₘₐₓ of about 30 min, about 50 min, about 70 min, about 90 min, about 110 min, about 130 min, about 150 min, about 170 min, about 190 min, about 210 min, about 230 min, about 250 min, about 270 min, or about 290 min.

Another embodiment described herein is a pharmaceutical composition, wherein the composition exhibits an in vitro dissolution rate comprising about 10% to about 99% dissolution after about 6 minutes to about 20 minutes at pH 7.2, including each integer within the specified rages of dissolution and time. In one aspect, the in vitro dissolution rate at pH 7.2 is about 10% after about 5.5 minutes. In one aspect, the in vitro dissolution rate at pH 7.2 is about 25% after about 7 minutes. In one aspect, the in vitro dissolution rate at pH 7.2 is about 50% after about 9 minutes. In another aspect, the in vitro dissolution rate at pH 7.2 is about 100% after about 20 minutes.

In another embodiment described herein, the oral pharmaceutical composition described herein is contained and dispensed from a tamper evident packaging. The term “tamper evident” or “tamper resistant” refers to a packaging of any kind that readily displays or permits an individual to observe any physical interference or manipulation of said packaging. The tamper evident packaging provides reasonable evidence to consumers that tampering has occurred. The tamper evident packaging additionally contains appropriate labelling statements describing the features and evidences of the tamper evident packaging. In one aspect, the tamper evident packaging comprises: bottles, film wrappers, blister or strip packs, bubble packs, heat shrink bands or wrappers, foil, paper, or plastic pouches, container mouth inner seals, tape seals, breakable caps, sealed metal tubes or plastic heat-sealed tubes, sealed cartons, aerosol containers, cans including metal and composite materials, or any combination thereof. The packaging may also comprise a dessicant and packing filler material to prevent the contents from shifting or rattling. The packaging may also contain appropriate instructions for prescribing, instructions for use, warnings, or other appropriate information.

It will be readily apparent to one of ordinary skill in the relevant arts that suitable modifications and adaptations to the compositions, methods, and applications described herein can be made without departing from the scope of any embodiments or aspects thereof. The compositions and methods provided are exemplary and are not intended to limit the scope of the specified embodiments. All of the various embodiments, aspects, and options disclosed herein can be combined in all variations. The scope of the compositions, formulations, methods, and processes described herein include all actual or potential combinations of embodiments, aspects, options, examples, and preferences herein described. All patents and publications cited herein are incorporated by reference herein for the specific teachings thereof.

### EXAMPLES

### Example 1

Exemplary immediate release matrix fill compositions useful for producing immediate release soft gel capsules as described herein are shown in Table 4. Composition components are set forth by weight percentage of the total weight of the gel mass composition. Such compositions may be encapsulated in soft capsules as described herein. The composition provides 200 mg of ibuprofen as a sodium salt.

TABLE 4
Exemplary Immediate Release Matrix Fill Composition

| Ingredient | mg/capsule | % weight |
|---|---|---|
| Polyethylene glycol 600 | 500.8 | 58.2 |
| Propylene glycol | 34.4 | 4.0 |
| Lactic acid | 68.8 | 8.0 |
| Ibuprofen, sodium salt | 256.0 | 29.8 |
| TOTAL | 860 mg | 100% |

Components and Relational Ratios

| | |
|---|---|
| Total Hydrophilic Vehicle | 62.23 |
| Excipient Matrix Fill | 70.23 |
| Ratio of PEG to Propylene glycol | 14.56 |
| Ratio of pH modifier to vehicle | 0.13 |
| Ratio of API to pH modifier | 3.72 |
| Ratio of API to Excipient Matrix Fill | 2.36 |

### Example 2

Exemplary immediate release matrix fill compositions useful for producing immediate release soft gel capsules as described herein are shown in Table 5. Composition components are set forth by weight percentage of the total weight of the gel mass composition. Such compositions may be encapsulated in soft capsules as described herein. The composition provides 200 mg of ibuprofen as a sodium salt.

TABLE 5
Exemplary Immediate Release Matrix Fill Composition

| Ingredient | mg/capsule | % weight |
|---|---|---|
| Polyethylene glycol 600 | 344.8 | 39.2 |
| Polyethylene glycol 400 | 200.0 | 22.7 |
| Propylene glycol | 26.4 | 3.0 |
| Lactic acid | 52.8 | 6.0 |
| Ibuprofen, sodium salt | 256.0 | 29.1 |
| TOTAL | 880 mg | 100% |

Components and Relational Ratios

| | |
|---|---|
| Total Hydrophilic Vehicle | 64.0 |
| Excipient Matrix Fill | 70.0 |
| Ratio of PEG 600 to PEG 400 | 1.77 |
| Ratio of pH modifier to vehicle | 0.09 |
| Ratio of API to pH modifier | 4.83 |
| Ratio of API to Excipient Matrix Fill | 2.41 |

### Example 3

The process for preparing an immediate-release matrix includes preparing a composition of one or more hydrophilic vehicles, one or more cosolvents, one or more pH modifiers, optionally, and one or more active pharmaceutical ingredients by heating said mixture from between 45° C. and 80° C. with stirring or agitation in a suitable vessel. Prior to encapsulation in a soft gel capsule described herein, the matrix is deaerated at a temperature of about 25° C. to about 45° C.

The process for manufacturing a soft capsule comprising the pharmaceutical composition as described herein includes preparing a gel mass for a soft capsule; casting the gel mass into films or ribbons using heat-controlled drums or surfaces; and manufacturing a soft capsule comprising a matrix fill using rotary die technology. During this process, the immediate-release matrix is injected in to the lumen as the soft capsule is formed by rotary die encapsulation.

### Example 4

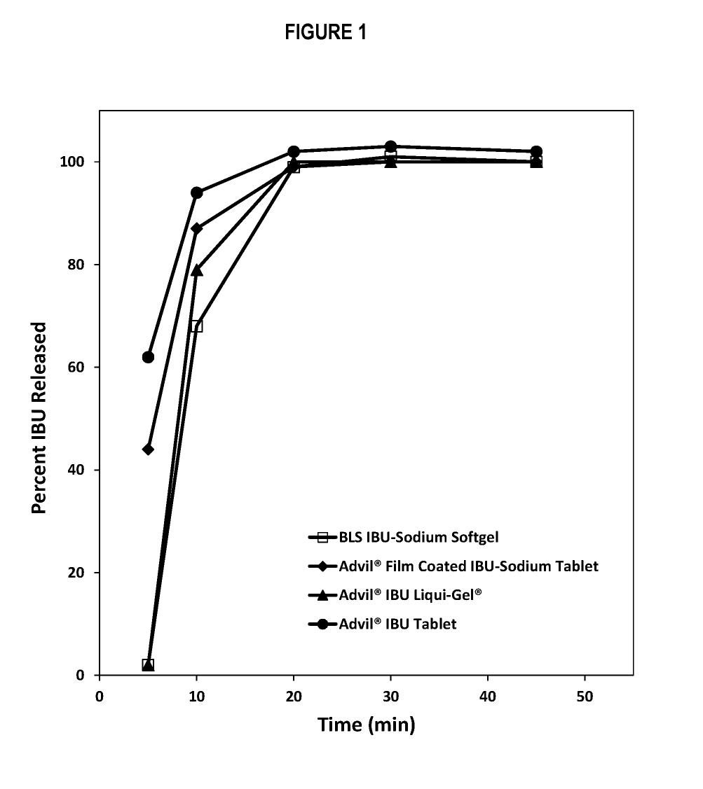

Soft gel capsules comprising the pharmaceutical composition shown in Table 5 were compared to reference drugs (ibuprofen free acid tablets, ibuprofen free acid liquigels, and film coated tablets of ibuprofen sodium) in dissolution experiments under USP Dissolution Apparatus 1 (basket) conditions in 900 mL 0.05 M phosphate buffer (pH 7.2) at 150 RPM at 37° C. Results are shown in Table 6 and FIG. 1. The soft gel compositions comprising ibuprofen sodium described herein have an approximately 5-minute lag time for dissolution of the capsule shell (similar to the ibuprofen free acid liquigels) compared to the ibuprofen free acid tablets and ibuprofen sodium film-coated tablets. The pharmaceutical composition described herein (BLS soft gel capsules comprising 256 mg of IBU sodium) releases substantially all of the ibuprofen after about 20 minutes in vitro at 37° C. in 900 mL of 50 mM phosphate buffer (pH 7.2).

TABLE 6
Percent Dissolution of Ibuprofen Compositions
Compared With Existing Products

| | Ibuprofen Released (%) | | | |
|---|---|---|---|---|
| | BLS Soft Gel | | Advil ® | Advil ® |
| | Capsules | Advil ® | Liqui-Gels | Film-Coated |
| | Ibuprofen | Ibuprofen, | Ibuprofen | Tablets |
| Time | Sodium, | Tablets, | 200 | Ibuprofen Sodium, |
| (min) | 256 mg | 200 mg | mg | 256 mg |
| 5.0 | 2.0 | 62.0 | 2.0 | 44.0 |
| 10.0 | 68.0 | 94.0 | 79.0 | 87.0 |
| 20.0 | 99.0 | 102.0 | 100.0 | 99.0 |
| 30.0 | 101.0 | 103.0 | 100.0 | 100.0 |
| 45.0 | 100.0 | 102.0 | 100.0 | 100.0 |

### Example 5

Soft gel capsules comprising the pharmaceutical compositions shown in Tables 4 and 5 were subjected to accelerated stability experiments at 1 month at 25° C./60% relative humidity (RH); 1 month at 30° C./65% RH, and 1 month at 40° C./70% RH. The results are shown in Table 7.

TABLE 7
Stability Data for Soft Gel Immediate Release IBU Compositions

| | Composition | Composition |
|---|---|---|
| Conditions | 1 (Table 4) | 2 (Table 5) |
| 1 month at 25° C./60% RH | Pass | Pass |
| 1 month at 30° C./65% RH | Pass | Pass |
| 1 month at 40° C./70% RH | Marginal | Pass |

### Example 6

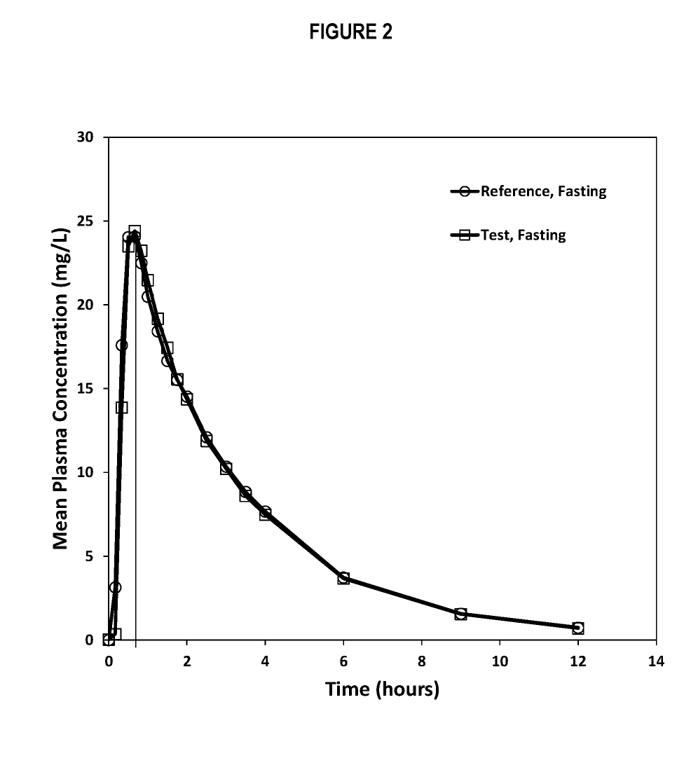

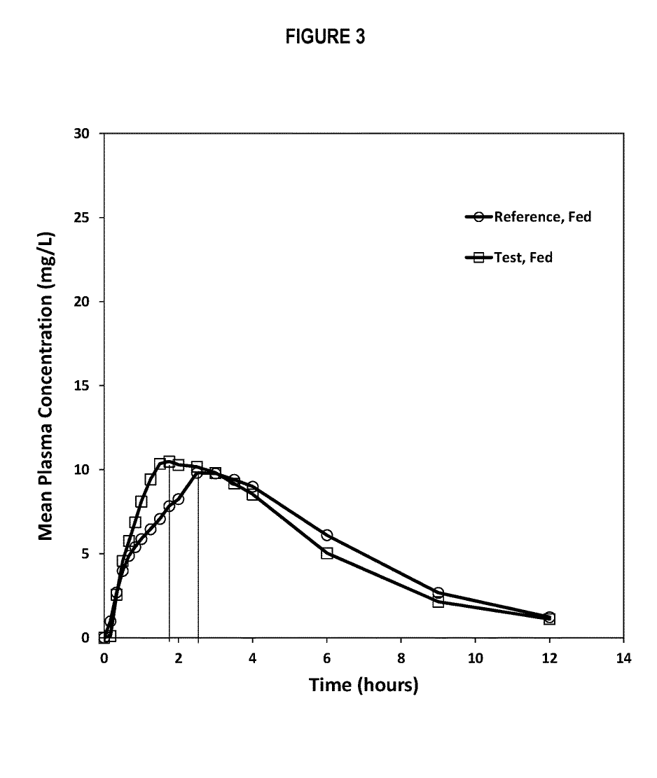

A randomized, crossover fasting and feed pharmacokinetic study was performed in 28 subjects using soft gel capsules comprising the pharmaceutical composition shown in Table 5 (i.e., Test) compared to a reference drug comprising a film-coated ibuprofen sodium tablet (i.e., Reference). The mean plasma concentrations of ibuprofen under fasting conditions are shown in Table 8 and FIG. 2. The mean plasma concentrations of ibuprofen under fed conditions are shown in Table 8 and FIG. 3. The pharmacokinetic parameters obtained from these studies are shown in Tables 9-14.

TABLE 8
Mean Plasma Concentrations of IBU under Fasting and Fed Conditions

| | Mean Plasma Concentrations of IBU (mg/L) | | | |
|---|---|---|---|---|
| | Test | | Reference | |
| | BLS Sof Gel Capsules | | Film-Coated Tablets | |
| Time | Ibuprofen sodium, 256 mg | | Ibuprofen Sodium 256 mg | |
| (hours) | Fasting | Fed | Fasting | Fed |
| 0.00 | 0.00 | 0.00 | 0.03 | 0.00 |
| 0.17 | 3.14 | 0.98 | 0.36 | 0.10 |
| 0.33 | 17.59 | 2.70 | 13.88 | 2.56 |
| 0.50 | 24.03 | 3.98 | 23.50 | 4.58 |
| 0.67 | 24.06 | 4.88 | 24.40 | 5.76 |
| 0.83 | 22.49 | 5.39 | 23.23 | 6.87 |
| 1.00 | 20.48 | 5.87 | 21.47 | 8.10 |
| 1.25 | 18.42 | 6.46 | 19.17 | 9.43 |
| 1.50 | 16.65 | 7.07 | 17.45 | 10.35 |
| 1.75 | 15.51 | 7.83 | 15.55 | 10.49 |
| 2.00 | 14.53 | 8.24 | 14.36 | 10.29 |
| 2.50 | 12.10 | 9.81 | 11.89 | 10.17 |
| 3.00 | 10.34 | 9.78 | 10.22 | 9.81 |
| 3.50 | 8.84 | 9.41 | 8.60 | 9.17 |
| 4.00 | 7.67 | 8.99 | 7.48 | 8.53 |
| 6.00 | 3.73 | 6.10 | 3.67 | 5.03 |
| 9.00 | 1.58 | 2.67 | 1.55 | 2.13 |
| 12.00 | 0.74 | 1.22 | 0.70 | 1.11 |

A pharmaceutical composition described herein (see, e.g., Table 5) had a median Tₘₐₓ of 0.667 h (˜40 min) a Cₘₐₓ of 28.5 mg/L under fasting conditions and a Tₘₐₓ of 1.75 h (˜105 min) a Cₘₐₓ of 14.86 mg/L under fed conditions. The average Tₘₐₓ among fasting and fed conditions is 1.20 h (˜72.5 min) and the average Cₘₐₓ among fasting and fed conditions is 21.7 mg/L.

TABLE 9
Summary of Test Bioavailability Studies for Ibuprofen
Randomized, 4-way crossover, open-label, single-dose, fasting and fed design
Test Mean Parameters (±SD) (% CV)

| | | | | | |
|---|---|---|---|---|---|
| | | AUC_{0→ 12h} | AUC_{0→ ∞} | | |
| Tₘₐₓ (hr)^{#} | Cₘₐₓ (mg/L) | (h · mg/L) | (h · mg/L) | t½ (h) | λ_{z} (1/h) |

Fasting Conditions

| | | | | | |
|---|---|---|---|---|---|
| 0.667 | 28.457 ± | 76.719 ± | 79.298 ± | 2.398 ± | 0.296 ± |
| (0.333-1.500) | 6.2915 (22.1) | 14.7641 (19.2) | 15.7163 (19.8) | 0.3901 (16.3) | 0.0430 (14.5) |

Test: Ibuprofen Capsules, 200 mg (provided as Ibuprofen Sodium 256 mg); Dose: 200 mg;
Dosage Form: Capsule; Route: Oral
Fed Conditions

| | | | | | |
|---|---|---|---|---|---|
| 1.750 | 14.863 ± | 62.306 ± | 66.164 ± | 2.331 ± | 0.311 ± |
| (0.500-6.000) | 4.7892 (32.2) | 12.0817 (19.4) | 13.9839 (21.1)* | 0.5230 (22.4)* | 0.0659 (21.2)* |

Test: Ibuprofen Capsules, 200 mg (provided as Ibuprofen Sodium 256 mg); Dose: 200 mg;
Dosage Form: Capsule Route: Oral
^{#}Tₘₐₓ is represented as median (min-max) value.
\hat{ }N = 27 and *N = 2 6.
Note 1:
Subject No.1003 (Period-IV, Reference Product-R2) had pre-dose concentration > 5% of Cₘₐₓ. Hence, the data for the same was excluded from the calculation of descriptive statistics for Reference Product-R2.
Note 2:
Subject No 1010 (Period-I and III) and 1021 (Period-II) had AUC_%Extrap_obs > 20% and hence, the primary pharmacokinetic parameter (i.e., AUC_{0→ ∞}) and secondary pharmacokinetic parameters [i.e., t½ and λ_{z}] were excluded from the calculation of descriptive statistics.

TABLE 10
Summary of Reference Bioavailability Studies for Ibuprofen
Randomized, 4-way crossover, open-label, single-dose, fasting and fed design
Reference Mean Parameters (±SD) (% CV)

| | | | | | |
|---|---|---|---|---|---|
| | | AUC_{0→ 12h} | AUC_{0→ ∞} | | |
| Tₘₐₓ (hr)^{#} | Cₘₐₓ (mg/L) | (h · mg/L) | (h · mg/L) | t½ (h) | λ_{z} (1/h) |

Fasting Conditions

| | | | | | |
|---|---|---|---|---|---|
| 0.500 | 26.818 ± | 77.952 ± | 80.653 ± | 2.399 ± | 0.297 ± |
| (0.333-1.750) | 5.7162 (21.3) | 15.9488 (20.5) | 16.9663 (21.0) | 0.3910 (16.3) | 0.0494 (16.6) |

Reference: Advil ® (buprofen) Tablets, 200 mg (provided as ibuprofen sodium, 256 mg); Dose: 200 mg;
Dosage Form: Tablet; Route: Oral
Fed Conditions

| | | | | | |
|---|---|---|---|---|---|
| 2.500 | 13.110 ± | 63.463 ± | 68.024 ± | 2.398 ± | 0.301 ± |
| (0.667-6.000)\hat{ } | 3.8651 (29.5)\hat{ } | 10.0331 (15.8)\hat{ } | 11.9320 (17.5)* | 0.4859 (20.3)* | 0.0617 (20.5)* |

Reference: Advil ® (ibuprofen) Tablets, 200 mg (provided as ibuprofen sodium, 256 mg); Dose: 200 mg;
Dosage Form: Tablet; Route: Oral
the data for the same was excluded from the calculation of descriptive statistics for Reference Product-R2.
>20% and hence, the primary
^{#}Tₘₐₓ is represented as median (min-max) value.
\hat{ }N = 27 and *N = 26.
Note 1:
Subject No.1003 (Period-IV, Reference Product-R2) had pre-dose concentration > 5% of Cₘₐₓ. Hence, the data for the same was excluded from the calculation of descriptive statistics for Reference Product-R2.
Note 2:
Subject No 1010 (Period-I and III) and 1021 (Period-II) had AUC_%Extrap_obs > 20% and hence, the primary pharmacokinetic parameter (i.e., AUC_{0→ ∞}) and secondary pharmacokinetic parameters [i.e., t½ and λ_{z}] were excluded from the calculation of descriptive statistics.

TABLE 11
Statistical Summary of the Comparative Bioavailability Data for
Unscaled Average BE Studies of Ibuprofen (Test 1 vs. Ref. 1)
Least Squares Geometric Means, Ratio of Means and 90%
Confidence Intervals
Fasting Bioequivalence Study

| | Test | | Reference | | | |
|---|---|---|---|---|---|---|
| | under fasting | | under fasting | | | |
| | conditions | | conditions | | Ratio | |
| Parameter | (T1) | N | (R1) | N | (%) | 90% C.I. |
| AUC_{0→T} | 75.287 | 28 | 76.388 | 28 | 98.56 | 96.35-100.82 |
| AUC_{0→∞} | 77.732 | 28 | 78.932 | 28 | 98.48 | 96.26-100.75 |
| Cₘₐₓ | 27.792 | 28 | 26.201 | 28 | 106.07 | 97.72-115.14 |

Ibuprofen Capsules (No. of subjects completed, N = 28) Dose (1 × 200 mg)

TABLE 12
Statistical Summary of the Comparative Bioavailability Data for
Unscaled Average BE Studies of Ibuprofen (Test 2 vs. Test 1)
Least Squares Geometric Means, Ratio of Means and 90%
Confidence Intervals

| | Test | | Test | | | |
|---|---|---|---|---|---|---|
| | under fed | | under fasting | | | |
| | conditions | | conditions | | Ratio | |
| Parameter | (T2) | N | (T1) | N | (%) | 90% C.I. |
| AUC_{0→T} | 61.154 | 28 | 75.287 | 28 | 81.23 | 78.08-84.50 |
| AUC_{0→∞} | 65.308 | 26 | 77.732 | 28 | 84.02 | 81.08-87.06 |
| Cₘₐₓ | 14.090 | 28 | 27.792 | 28 | 50.70 | 46.32-55.49 |

Ibuprofen Capsules (No. of subjects completed, N = 28) Dose (1 × 200 mg)

TABLE 13
Statistical Summary of the Comparative Bioavailability Data for
Unscaled Average BE Studies of Ibuprofen (Ref. 2 vs. Ref. 1)
Least Squares Geometric Means, Ratio of Means and 90%
Confidence Intervals

| | Reference | | Reference | | | |
|---|---|---|---|---|---|---|
| | under fed | | under fasting | | | |
| | conditions | | conditions | | Ratio | |
| Parameter | (R2) | N | (R1) | N | (%) | 90% C.I. |
| AUC_{0→T} | 62.610 | 27 | 77.273 | 27 | 81.03 | 78.34-83.80 |
| AUC_{0→∞} | 66.839 | 26 | 79.876 | 27 | 83.68 | 81.27-86.16 |
| Cₘₐₓ | 12.675 | 27 | 26.441 | 27 | 47.94 | 43.57-52.74 |

(Excluding Subject No. 1003 whose pre-dose concentration was >5% of Cₘₐₓ)
Ibuprofen Capsules (No. of subjects completed, N = 27) Dose (1 × 200 mg)

TABLE 14
Pharmacokinetic Parameters and % Ratio of Test vs. Reference

| | | Form | | | | 90% CI | 90% CI |
|---|---|---|---|---|---|---|---|
| | Depend. | Ref. | Test | Param. | IBU_{TEST}/IBU_{REF} % | Low | High |
| Fasting | Ln(Cₘₐₓ) | r1 | t1 | Cₘₐₓ | 106.1 | 97.7 | 115.1 |
| | Ln(AUC_{L}) | r1 | t1 | AUC_{L} | 98.5 | 96.3 | 100.8 |
| | Ln(AUC_{inf}) | r1 | t1 | AUC_{INF} | 98.5 | 96.2 | 100.7 |
| Fed | Ln(Cₘₐ) | r2 | t2 | Cₘₐₓ | 111.9 | 101.6 | 123.2 |
| | Ln(AUC_{L}) | r2 | t2 | AUC_{L} | 98.4 | 95.1 | 101.8 |
| | Ln(AUC_{inf}) | r2 | t2 | AUC_{INF} | 98.6 | 95.8 | 101.4 |
| Fed/Fasting (Test) | Ln(Cₘₐ) | t1 | t2 | Cₘₐₓ | 50.7 | 46.3 | 55.5 |
| | Ln(AUC_{L}) | t1 | t2 | AUC_{L} | 81.2 | 78.1 | 84.5 |
| | Ln(AUC_{inf}) | t1 | t2 | AUC_{INF} | 84.9 | 82.1 | 87.9 |
| Fed/Fasting (Ref.) | Ln(Cₘₐ) | r1 | r2 | Cₘₐₓ | 47.9 | 43.6 | 52.7 |
| | Ln(AUC_{L}) | r1 | r2 | AUC_{L} | 81.0 | 78.3 | 83.8 |
| | Ln(AUC_{inf}) | r1 | r2 | AUC_{INF} | 85.0 | 82.3 | 87.8 |

TABLE 15
Tₘₐₓ Comparison Between Test and Reference
Under Fasting and Fed Conditions

| Parameter | Mean (h) | Minimum | (h) | Median (h) | Max (h) |
|---|---|---|---|---|---|
| Ref. (Fasting) | Tₘₐₓ | 0.64 | 0.33 | 0.50 | 1.75 |
| Ref. (Fed) | Tₘₐₓ | 2.86 | 0.67 | 2.5 | 6.0 |
| Test (Fast) | Tₘₐₓ | 0.64 | 0.33 | 0.67 | 1.5 |
| Test (Fed) | Tₘₐₓ | 2.27 | 0.50 | 1.75 | 6.0 |

The soft gel capsules containing the pharmaceutical compositions described herein (e.g., Table 5; Test) are bioequivalent to the reference drug comprising a film-coated ibuprofen sodium tablet, Reference. See Tables 8-15. The soft gel pharmaceutical composition has significantly more rapid absorption of ibuprofen under fed conditions as compared to the reference: Tₘₐₓ Test Fed: 1.750 h (105 min); Tₘₐₓ Ref. Fed: 2.5 h (150 min); Δ_{|Avg. Test-Avg. Ref.|}=0.75 h (45 min). The Tₘₐₓ for the soft gel pharmaceutical composition described herein is comparable to the reference under fasting conditions: Tₘₐₓ Test Fasting: 0.667 h (40 min); Tₘₐₓ Ref. Fasting: 0.5 h (30 min). Under normal usage by a subject, food is likely to be ingested prior to, shortly after, or contemporaneously with the pharmaceutical composition described herein. Accordingly, the Tₘₐₓ for fed conditions is highly relevant to subjects for the perceptible relief of pain, inflammation diminution, or fever diminution. This leads to a quicker time to maximal therapeutic efficacy. Subjects may achieve perceptible pain relief approximately 0.75 h (45 min) faster than the comparable reference dosage under fed conditions.

### Example 7

Exemplary immediate release matrix compositions useful for producing immediate release soft gel capsules as described herein are shown in Table 16. Composition components are set forth by weight percentage of the total weight of the gel mass composition. Such compositions may be encapsulated in soft capsules as described herein.

TABLE 16
Exemplary Immediate Release Matrix Compositions

| | Weight Percentage (%) | | | | | |
|---|---|---|---|---|---|---|
| Ingredient | EX 1 | EX 2 | EX 3 | EX 4 | EX 5 | EX 6 |
| Polyethylene glycol 600 | 65.0 | 26.0 | 61.0 | 32.0 | 40.0 | 35.0 |
| Polyethylene glycol 400 | 8.0 | 50.0 | — | 30.0 | 15.0 | 40.0 |
| Propylene glycol | 3.0 | 8.0 | 3.0 | 3.0 | — | 5.0 |
| pH modifier | 4.0 | 6.0 | 7.0 | 6.0 | 5.0 | 10.0 |
| Ibuprofen Sodium (IBU Na) | 20.0 | 10.0 | 29.0 | 29.0 | 40.0 | 10.0 |
| TOTAL | 100 | 100 | 100 | 100 | 100 | 100 |

Components and Relational Ratios

| | | | | | | |
|---|---|---|---|---|---|---|
| Total Hydrophilic Vehicle | 76.0 | 84.0 | 64.0 | 65.0 | 55.0 | 80.0 |
| Excipient Matrix Fill | 80.0 | 90.0 | 71.0 | 71.0 | 60.0 | 90.0 |
| Ratio of PEG 600 to PEG 400 | 8.13 | 0.52 | — | 1.07 | 2.67 | 0.88 |
| Ratio of pH modifier to vehicle | 0.05 | 0.07 | 0.11 | 0.09 | 0.09 | 0.13 |
| Ratio of API to pH modifier | 5.00 | 1.67 | 4.14 | 4.83 | 8.00 | 1.00 |
| Ratio of API to Excipient Matrix Fill | 4.00 | 9.00 | 2.45 | 2.45 | 1.50 | 9.00 |

What is claimed is:

1. An oral immediate release pharmaceutical composition comprising a soft capsule encapsulating a matrix comprising:
(a) at least one hydrophilic vehicle;
(b) at least one co-solvent;
(c) at least one pH modifier; and
(d) one or more active pharmaceutical ingredients;
wherein the ratio of the active pharmaceutical ingredient to the pH modifier is about 3.5:1 to about 5.5:1.

2. The composition of claim 1, wherein the composition releases substantially all of the one or more active pharmaceutical ingredients after about 20 minutes in vitro.

3. The composition of claim 1, wherein the matrix comprises:
(a) about 30-70% of at least one hydrophilic vehicle;
(b) about 3-5% of at least one co-solvent;
(c) about 3-10% of at least one pH modifier; and
(d) about 20-40% of one or more active pharmaceutical ingredients.

4. The composition of claim 1, wherein the hydrophilic vehicle comprises one or more polyethylene glycols.

5. The composition of claim 1, wherein the co-solvent comprises propylene glycol, glycerol, or a combination thereof.

6. The composition of claim 1, wherein the pH modifier comprises acetic acid, lactic acid, malic acid, citric acid, tartaric acid, or a combination thereof.

7. The composition of claim 1, wherein the pH modifier comprises lactic acid.

8. The composition of claim 1, wherein the composition comprises a pH modifier comprising lactic acid at a weight percentage up to about 8%.

9. The composition of claim 1, wherein the active pharmaceutical ingredient comprises one or more of aspirin, ibuprofen, aceclofenac, acemetacin, aloxiprin, azapropazone, benorilate, bromfenac, carprofen, celecoxib, choline magnesium salicylate, diclofenac, diflunisal, etodolac, etoricoxib, faislamine, fenbufen, fenoprofen, flurbiprofen, indometacin, ketoprofen, ketorolac, lornoxicam, loxoprofen, meloxicam, meclofenamic acid, mefenamic acid, meloxicam, metamizole, methyl salicylate, magnesium salicylate, nabumetone, naproxen, nimesulide, oxyphenbutazone, parecoxib, phenylbutazone, piroxicam, salicyl salicylate, sulindac, sulfinpyrazone, suprofen, tenoxicam, tiaprofenic acid, tolmetin, valdecoxib, salts thereof, or combinations thereof.

10. The composition of claim 1, wherein the active pharmaceutical ingredient comprises a salt form of ibuprofen.

11. The composition of claim 1, wherein the active pharmaceutical ingredient comprises about 260 mg of ibuprofen sodium.

12. The composition of claim 1, wherein the ratio of the active pharmaceutical ingredient to the pH modifier is about 5:1.

13. The composition of claim 1, wherein the ratio of the active pharmaceutical ingredient to hydrophilic vehicle and co-solvent is about 1:2 to about 1:4.

14. The composition of claim 1, wherein the ratio of the active pharmaceutical ingredient to a combined weight percentage of the hydrophilic vehicle, co-solvent, and pH modifier is about 1:2 to about 1:3.

15. The composition of claim 1, wherein the active pharmaceutical ingredient comprises a salt form of ibuprofen and one or more of a cold, cough, allergy, decongestant, antitussive, expectorant, antihistamine, stimulant, sedative, anti-inflammatory, antibiotic, anti-viral, anti-asthmatic, anti-migraine, hypnotic, narcotic analgesic, or narcotic antagonist active pharmaceutical ingredients.

16. The composition of claim 1, wherein the active pharmaceutical ingredient comprises a salt form of ibuprofen and one or more of astemizole, azelastine, azatadine, brompheniramine, carbinoxamine, cetirizine, chlorpheniramine, clemastine, cyproheptadine, desloratadine, dexbrompheniramine, dexchlorpheniramine, diphenhydramine, fexofenadine, hydroxyzine, levocetirizine, loratadine, phenindamine, pheniramine, phenyltoloxamine, promethazine, pyrilamine, terfenadine, tripelennamine, triprolidine, acetyl dihydrocodeine, benproperine, benzonatate, benzylmorphine, bibenzonium bromide, butamirate, butorphanol, carbetapentane, chlophedianol, clobutinol, clofedanol, cloperastine, codeine, dextromethorphan, diacetylmorphine, dibunate, dihydrocodeine, dimemorfan, dimethoxanate, diphenhydramine, dropropizine, droxypropine, ethylmorphine, fedrilate, glaucine, hydrocodone, hydromorphone, isoaminile, laudanum, levodropropizine, levomethadone, levopropoxyphene, meprotixol, methadone, morclofone, nepinalone, nicocodine, nicodicodine, normethadone, noscapine, oxeladin, oxolamine, pentoxyverine, pholcodine, pipazetate, piperidione, prenoxdiazine, tipepidine, zipeprol, acetylcysteine, althea root, ambroxol, antimony pentasulfide, bromhexine, carbocisteine, cineole, combinations, combinations, creosote, dembrexine hydrochloride, domiodol, dornase alfa, eprazinone, erdosteine, guaiacolsulfonate, guaifenesin, hederae helicis folium, ipecacuanha, letosteine, levo verbenone, mannitol, mesna, neltenexine, potassium iodide, senega, sobrerol, stepronin, tiopronin, tyloxapol, or a mixture or combination thereof.

17. The composition of claim 1, wherein the matrix comprises:
(a) about 39% polyethylene glycol 600;
(b) about 23% polyethylene glycol 400;
(c) about 3% propylene glycol;
(d) about 6% lactic acid; and
(e) about 30% ibuprofen sodium salt.

18. The composition of claim 1, wherein the composition provides one or more of the following pharmacokinetic parameters:
(a) a mean plasma ibuprofen Tₘₐₓ of about 0.7 hours to about 1.8 hours;
(b) a mean plasma ibuprofen Cₘₐₓ of about 15 mg/L to about 29 mg/L;
(c) a mean plasma ibuprofen AUC_{0→12 h} of about 62 h·mg/L to about 77 h·mg/L;
(d) a mean plasma ibuprofen AUC_{0→∞} of about 66·mg/L to about 79·mg/L;
(e) a mean ibuprofen half-life (t½) of about 2.3 h to about 2.4 h; or
(f) a mean ibuprofen terminal elimination rate constant (λ_{z}) of about 0.29 h⁻¹ to about 0.31 h⁻¹.

19. The composition of claim 1, wherein the composition provides one or more of the following pharmacokinetic parameters:
(a) a mean plasma ibuprofen Tₘₐₓ of about 0.7 hours under fasting conditions;
(b) a mean plasma ibuprofen Cₘₐₓ of about 29 mg/L under fasting conditions;
(c) a mean plasma ibuprofen AUC_{0→12 h} of about 77 h·mg/L under fasting conditions;
(d) a mean plasma ibuprofen AUC_{0→∞} of about 79·mg/L under fasting conditions;
(e) a mean ibuprofen half-life (t½) of about 2.4 h under fasting conditions; or
(f) a mean ibuprofen terminal elimination rate constant (λ_{z}) of about 0.29 h⁻¹ under fasting conditions;
or
(g) a mean plasma ibuprofen Tₘₐₓ of about 1.8 hours under fed conditions;
(h) a mean plasma ibuprofen Cₘₐₓ of about 15 mg/L under fed conditions;
(i) a mean plasma ibuprofen AUC_{0→12 h} of about 62 h·mg/L under fed conditions;
(j) a mean plasma ibuprofen AUC_{0→∞} of about 66·mg/L under fed conditions;
(k) a mean ibuprofen half-life (t½) of about 2.3 h under fed conditions; or
(l) a mean ibuprofen terminal elimination rate constant (λ_{z}) of about 0.31 h⁻¹ under fed conditions.

20. The composition of claim 1, wherein the composition is useful for treating, retarding the progression of, delaying the onset of, prophylaxis of, amelioration of, or reducing the symptoms of pain, inflammation, or fever.

21. The composition of claim 1, wherein the composition is useful for treating, retarding the progression of, delaying the onset of, prophylaxis of, amelioration of, or reducing the symptoms of pain, inflammation, or fever, including but not limited to, bacterial or viral infections, osteoarthritis, rheumatoid arthritis, tendonitis, bursitis, chronic neuropathies, shingles, chronic sports injuries, chronic malignancies and/or cancer, radiculopathy, sciatica, kidney stones, menstrual pain or inflammation, dysmenorrhea, endometriosis, headache, acute migraines, ankylosing spondylitis, spondylarthritis, or gout.

22. The composition of claim 1, wherein the soft capsule comprises:
(a) about 25-50% of a film-forming polymer;
(b) about 15-25% of a plasticizer;
(c) about 20-40% of a solvent;
(d) optionally, about 0.05-0.1% of an coloring agent; and
(e) optionally, about 0.5-1.5% of an opacifier.

23. The composition of claim 1, wherein the soft capsule comprises:
(a) about 40% of at least one film-forming polymer;
(b) about 20% of at least one plasticizer;
(c) about 36% of a solvent; and
(d) optionally, about 0.1% of a coloring agent;

24. The composition of claim 1, wherein the soft capsule comprises:
(a) about 43% gelatin;
(b) about 20% glycerol; and
(c) about 36% water.

25. An immediate release pharmaceutic composition comprising a soft capsule encapsulating a matrix fill, the soft capsule comprising:
(a) about 40% of at least one film-forming polymer;
(b) about 20% of at least one plasticizer;
(c) about 36% of a solvent; and
(d) optionally, about 0.1% of a coloring agent; and
the matrix fill comprising:
(e) about 39% polyethylene glycol 600;
(f) about 23% polyethylene glycol 400;
(g) about 3% propylene glycol;
(h) about 6% lactic acid; and
(i) about 30% ibuprofen, sodium salt.

26. The composition of claim 25, wherein the composition releases essentially all of the ibuprofen after about 20 minutes in vitro.

26. The composition of claim 25, wherein the ibuprofen sodium comprises about 260 mg.

27. The composition of claim 25, wherein the composition provides one or more of the following pharmacokinetic parameters:
(a) a mean plasma ibuprofen Tₘₐₓ of about 0.7 hours to about 1.8 hours;
(b) a mean plasma ibuprofen Cₘₐₓ of about 15 mg/L to about 29 mg/L;
(c) a mean plasma ibuprofen AUC_{0→12 h} of about 62 h·mg/L to about 77 h·mg/L;
(d) a mean plasma ibuprofen AUC_{0→∞} of about 66·mg/L to about 79·mg/L;
(e) a mean ibuprofen half-life (t½) of about 2.3 h to about 2.4 h; or
(f) a mean ibuprofen terminal elimination rate constant (λ_{z}) of about 0.29 h⁻¹ to about 0.31 h⁻¹.

28. The composition of claim 25, wherein the composition provides one or more of the following pharmacokinetic parameters:
(a) a mean plasma ibuprofen Tₘₐₓ of about 0.7 hours under fasting conditions;
(b) a mean plasma ibuprofen Cₘₐₓ of about 29 mg/L under fasting conditions;
(c) a mean plasma ibuprofen AUC_{0→12 h} of about 77 h·mg/L under fasting conditions;
(d) a mean plasma ibuprofen AUC_{0→∞} of about 79·mg/L under fasting conditions;
(e) a mean ibuprofen half-life (t½) of about 2.4 h under fasting conditions; or
(f) a mean ibuprofen terminal elimination rate constant (λ_{z}) of about 0.29 h⁻¹ under fasting conditions;
or
(g) a mean plasma ibuprofen Tₘₐₓ of about 1.8 hours under fed conditions;
(h) a mean plasma ibuprofen Cₘₐₓ of about 15 mg/L under fed conditions;
(i) a mean plasma ibuprofen AUC_{0→12 h} of about 62 h·mg/L under fed conditions;
(j) a mean plasma ibuprofen AUC_{0→∞} of about 66·mg/L under fed conditions;
(k) a mean ibuprofen half-life (t½) of about 2.3 h under fed conditions; or
(l) a mean ibuprofen terminal elimination rate constant (λ_{z}) of about 0.31 h⁻¹ under fed conditions.

29. The composition of claim 25, wherein the composition is useful for treating, retarding the progression of, delaying the onset of, prophylaxis of, amelioration of, or reducing the symptoms of pain, inflammation, fever, or symptoms stemming from cough or cold.

30. The composition of claim 25, wherein the active pharmaceutical ingredient further comprises one or more of astemizole, azelastine, azatadine, brompheniramine, carbinoxamine, cetirizine, chlorpheniramine, clemastine, cyproheptadine, desloratadine, dexbrompheniramine, dexchlorpheniramine, diphenhydramine, fexofenadine, hydroxyzine, levocetirizine, loratadine, phenindamine, pheniramine, phenyltoloxamine, promethazine, pyrilamine, terfenadine, tripelennamine, triprolidine, acetyl dihydrocodeine, benproperine, benzonatate, benzylmorphine, bibenzonium bromide, butamirate, butorphanol, carbetapentane, chlophedianol, clobutinol, clofedanol, cloperastine, codeine, dextromethorphan, diacetylmorphine, dibunate, dihydrocodeine, dimemorfan, dimethoxanate, diphenhydramine, dropropizine, droxypropine, ethylmorphine, fedrilate, glaucine, hydrocodone, hydromorphone, isoaminile, laudanum, levodropropizine, levomethadone, levopropoxyphene, meprotixol, methadone, morclofone, nepinalone, nicocodine, nicodicodine, normethadone, noscapine, oxeladin, oxolamine, pentoxyverine, pholcodine, pipazetate, piperidione, prenoxdiazine, tipepidine, zipeprol, acetylcysteine, althea root, ambroxol, antimony pentasulfide, bromhexine, carbocisteine, cineole, combinations, combinations, creosote, dembrexine hydrochloride, domiodol, dornase alfa, eprazinone, erdosteine, guaiacolsulfonate, guaifenesin, hederae helicis folium, ipecacuanha, letosteine, levo verbenone, mannitol, mesna, neltenexine, potassium iodide, senega, sobrerol, stepronin, tiopronin, tyloxapol, or a mixture or combination thereof.

31. The composition of claim 25, wherein the composition is useful for treating, retarding the progression of, delaying the onset of, prophylaxis of, amelioration of, or reducing the symptoms of pain, inflammation, or fever, including but not limited to, bacterial or viral infections, osteoarthritis rheumatoid arthritis, tendonitis, bursitis, chronic neuropathies, shingles, chronic sports injuries, chronic malignancies and/or cancer, radiculopathy, sciatica, kidney stones, menstrual pain or inflammation, dysmenorrhea, endometriosis, headache, acute migraines, ankylosing spondylitis, spondylarthritis, or gout.

32. A method for delivering a 200 mg dose equivalent of ibuprofen free acid comprising administering to a subject ibuprofen sodium admixed with lactic acid and other pharmaceutically acceptable excipients in a soft gel capsule, the method capable of achieving one or more of the following pharmacokinetic parameters:
(a) a mean plasma ibuprofen Tₘₐₓ of about 0.7 hours to about 1.8 hours;
(b) a mean plasma ibuprofen Cₘₐₓ of about 15 mg/L to about 29 mg/L;
(c) a mean plasma ibuprofen AUC_{0→12 h} of about 62 h·mg/L to about 77 h·mg/L;
(d) a mean plasma ibuprofen AUC_{0→∞} of about 66·mg/L to about 79·mg/L;
(e) a mean ibuprofen half-life (t½) of about 2.3 h to about 2.4 h; or
(f) a mean ibuprofen terminal elimination rate constant (A′) of about 0.29 h⁻¹ to about 0.31 h⁻¹.

33. The method of claim 32, wherein the dose equivalent comprises about 260 mg of ibuprofen sodium.

34. The method of claim 32, wherein lactic acid comprises about 50 to about 60 mg.

35. The method of claim 32, wherein the pharmaceutically acceptable excipients comprise one or more polyethylene glycols, propylene glycol, or a combination thereof.

36. The method of claim 32, wherein the subject experiences relief from one or more of the symptoms of pain, inflammation, or fever.

37. A method for treating, retarding the progression of, delaying the onset of, prophylaxis of, amelioration of, or reducing the symptoms of pain, inflammation, fever, or symptoms stemming from cough or cold comprising the administration of a therapeutically effective amount of ibuprofen sodium comprising the pharmaceutical compositions described in claim 19.

38. The method of claim 37, wherein the therapeutically effective amount of ibuprofen sodium is about 260 mg.

39. The method of claim 37, wherein said administering the composition provides one or more of the following pharmacokinetic parameters:
(a) a mean plasma ibuprofen Tₘₐₓ of about 0.7 hours to about 1.8 hours;
(b) a mean plasma ibuprofen Cₘₐₓ of about 15 mg/L to about 29 mg/L;
(c) a mean plasma ibuprofen AUC_{0→12 h} of about 62 h·mg/L to about 77 h·mg/L;
(d) a mean plasma ibuprofen AUC_{0→∞} of about 66·mg/L to about 79·mg/L;
(e) a mean ibuprofen half-life (t½) of about 2.3 h to about 2.4 h; or
(f) a mean ibuprofen terminal elimination rate constant (λ_{z}) of about 0.29 h⁻¹ to about 0.31 h⁻¹.

40. The method of claim 37, wherein said administering the composition provides one or more of the following pharmacokinetic parameters:
(a) a mean plasma ibuprofen Tₘₐₓ of about 0.7 hours under fasting conditions;
(b) a mean plasma ibuprofen Cₘₐₓ of about 29 mg/L under fasting conditions;
(c) a mean plasma ibuprofen AUC_{0→12 h} of about 77 h·mg/L under fasting conditions;
(d) a mean plasma ibuprofen AUC_{0→∞} of about 79·mg/L under fasting conditions;
(e) a mean ibuprofen half-life (t½) of about 2.4 h under fasting conditions; or
(f) a mean ibuprofen terminal elimination rate constant (Δ_{z}) of about 0.29 h⁻¹ under fasting conditions;
or
(g) a mean plasma ibuprofen Tₘₐₓ of about 1.8 hours under fed conditions;
(h) a mean plasma ibuprofen Cₘₐₓ of about 15 mg/L under fed conditions;
(i) a mean plasma ibuprofen AUC_{0→12 h} of about 62 h·mg/L under fed conditions;
(j) a mean plasma ibuprofen AUC_{0→∞} of about 66·mg/L under fed conditions;
(k) a mean ibuprofen half-life (t½) of about 2.3 h under fed conditions; or
(l) a mean ibuprofen terminal elimination rate constant (λ_{z}) of about 0.31 h⁻¹ under fed conditions.

41. The method of claim 37, wherein the administration is sufficient to achieve a reduction of pain, reduction of inflammation, or reduction of fever relative to baseline in the subject without substantially inducing one or more of abdominal pain, acid or sour stomach, belching, bloating, cloudy urine, decrease in amount of urine, decrease in urine output or decrease in urine-concentrating ability, diarrhea, difficulty having a bowel movement (stool), excess air or gas in stomach or intestines, full feeling, heartburn, indigestion, itching skin, nausea, noisy, rattling breathing, pain or discomfort in chest, upper stomach, or throat, pale skin, passing gas, rash with flat lesions or small raised lesions on the skin, shortness of breath, swelling of face, fingers, hands, feet, lower legs, or ankles, troubled breathing at rest, troubled breathing with exertion, unusual bleeding or bruising, unusual tiredness or weakness, vomiting, or weight gain.
